(19) United States
(12) Patent Application Publication
(10) Pub. No.: US 2023/0137491 A1
(43) Pub. Date: May 4, 2023

(54) PEPTIDES AND USES THEREOF

(71) Applicant: SINTICA BIOTECH SOCIETÀ A RESPONSABILITÀ LIMITATA SEMPLIFICATA, Milano (MI) (IT)

(72) Inventors: Roberta LOTTI, Roncoferraro (MN) (IT); Elisabetta PALAZZO, Soliera (MO) (IT); Marika QUADRI, San Benedetto Po (MN) (IT); Alessandra MARCONI, Reggio Emilia (RE) (IT); Carlo PINCELLI, Modena (MO) (IT); Silvio TRAVERSA, Palazzo Canavese (TO) (IT)

(73) Assignee: SINTICA BIOTECH SOCIETÀ A RESPONSABILITÀ LIMITATA SEMPLIFICATA, Milano (MI) (IT)

(21) Appl. No.: 17/918,096

(22) PCT Filed: Apr. 9, 2021
(86) PCT No.: PCT/EP2021/059307

§ 371 Date: Oct. 10, 2022

(30) Foreign Application Priority Data
Apr. 10, 2020 (IT) 102020000007720

(51) Int. Cl. A61K 38/08; A61K 47/60; A61K 9/127; A61K 45/06; A61P 35/00; A61P 17/00

(52) U.S. Cl. CPC A61K 38/08; A61K 47/60; A61K 9/127; A61K 45/06; A61P 35/00; A61P 17/00

(57) ABSTRACT

The present invention relates to CD271-interacting peptides and derivatives thereof and to their use as a medicament for the treatment of CD271-related diseases, in particular for the treatment of melanoma and other CD271-related skin diseases.

### TECHNICAL FIELD

The present invention relates to CD271-interacting peptides and derivatives thereof and to their use as a medicament for the treatment of CD271-related diseases including neuroblastoma, glioblastoma, astrocytoma, head and neck squamous cell carcinoma and/or for the treatment of CD271-related skin diseases including melanoma, psoriasis, Merkel-cell carcinoma, chronic skin ulcers, cutaneous squamous cell carcinoma. In particular, the CD271-interacting peptides of the invention are for use in the treatment of melanoma.

### BACKGROUND ART

The nerve growth factor (NGF) receptor CD271 is expressed in the skin, where it participates in the so called “neurotrophin network” along with the high affinity receptor Trk. By acting alone or in combination with Trk in many skin cells (keratinocytes, melanocytes, fibroblasts), CD271 mediates a number of autocrine and paracrine functions. In keratinocytes, CD271 is instrumental in the transition from stem to transit amplifying cells. CD271 is also expressed in melanocytes and its levels change in melanoma, where the receptor seems to mediate invasiveness and migration. A “CD271-related disease” is defined as a disease in which the pathological state induces the expression or the overexpression of the CD271 receptor in the involved cells. A “CD271-related skin disease” is defined as a disease in which the pathological state induces the expression or the overexpression of the CD271 receptor in the involved cells, where the involved cells are located in the epidermis and/or derma and can be keratinocytes and/or melanocytes and/or fibroblasts.

Melanoma is a substantial public health problem, with an annual increasing incidence more rapid than any other type of cancer (Ali Z, Yousaf N, Larkin J. Melanoma epidemiology, biology and prognosis. EJC Suppl 11: 81-91, 2013; Lens M B, Dawes M. “Global perspectives of contemporary epidemiological trends of cutaneous malignant melanoma” Br. J. Dermatol., 2004; 150: 179-185) that makes it one of the most common forms of cancer in young adult (Jemal A, Siegel R, Ward E, Hao Y, Xu J, Thun M J. “Cancer statistics, 2005” CA, Cancer J Clin, 2005; 55: 10-29). The incidence of cutaneous malignant melanoma has risen by 3-7% on average over the last decades (de Vries E, Bray F I, Eggermont A M, Coebergh J W; “European Network of Cancer Registries. Monitoring stage-specific trends in melanoma incidence across Europe reveals the need for more complete information on diagnostic characteristics” Eur J Cancer Prey, 2004; 13: 387-95). Currently, it represents 1-3% of all malignant tumours, with Australia and New Zealand having the highest incidence (40 new cases/100000 inhabitants per year), followed by North European countries and the USA; on the other hand, Japan and central Africa share the lowest incidence (0.4/100000). In Italy, incidence trends have documented an annual increase of 6.2% in males and 5.8% in females from 1987 to 1997; the standardized incidence rates vary between 3.9 and 12.5/100000 males and between 3.2 and 13/100000 females. Mortality rates have increased as well, with 232.000 new cases of melanoma in 2012 and 55.000 deaths worldwide (Ferlay J, Soerjomataram I, Dikshit R, Eser S, Mathers C, Rebelo M, Parkin D M, Forman D and Bray F. Cancer incidence and mortality worldwide: sources, methods and major patterns in GLOBOCAN2012. Int J Cancer 2015; 136: E359-E386).

Up to one-fifth of patients progress to metastatic (stage IV) disease, with a median survival of 6 months and a 5-year survival rate of less than 5% (Cummins D L, Cummins J M, Pantle H, Silverman M A, Leonard A L, Chanmugam A. “Cutaneous malignant melanoma” Mayo Clin Proc, 2006; 81: 500-507). To date, the only FDA-approved chemotherapy for melanoma is the alkylating agent dacarbazine (DTIC), which gives clinical responses lasting 6-8 months in 5-10% of patients and cures in about 1% (Serrone L, Zeuli M, Sega F M, Cognetti F. “Dacarbazine-based chemotherapy for metastatic melanoma: thirty-year experience overview” J Exp Clin Res, 2000; 19: 21— 34). A number of immunotherapies are now available for the treatment of metastatic melanoma, FDA have recently approved the humanized monoclonal antibodies pembrolizumab and nivolumab which target Programmed Death Receptor 1 (PD-1), thus increasing the ability of the immune system to attack melanoma cells. A recent study reports an objective response rates of 33% and a median overall survival of 23 months in the total population, and 45% and 31 months in the treated patients. 44% of all responders had a response duration of at least 1 year with low toxicity (Kim D W, Zager J S, Eroglu Z. “improving clinical outcomes with pembrolizumab in patients with advanced melanoma” Chinese Cl Oncology 2017; 6: 1-4; Ribas A, Hamid O, Daud A et al. “Association of pembrolizumab with tumour response and survival among patients with advanced melanoma”. JAMA 2016; 315:1600-1609). Activity of nivolumab was reportedly similar with pembrolizumab with a 40% objective response rates (Robert C, Long G V, Brady B et al. “Nivolumab in previously untreated melanoma without BRAF mutation”. N Engl J Med 2015; 372:320-330). In addition, the humanized monoclonal antibody ipilimumab blocks the activity of CTLA-4, thus allowing T cells to activate and attack melanoma. Ipilimumab was shown to prolong survival in advanced melanoma (Eggermont A M, Chiarion-Sileni V, Grob J J et al. “Prolonged survival in stage III melanoma with ipilimumab adjuvant therapy. N Engl J Med 2016; 10:1845-1855). Finally, several targeted therapies have become available with the function of blocking the activity of some mutated forms of BRAF, that are critical for melanoma growth. In particular, vemurafenib is a kinase inhibitor that blocks V600E-mutated BRAF, which is present in half of patients with melanoma. Vemurafenib alone or in combination with other BRAF mutated inhibitors such as trametinib, cobimetinib or dabrafenib improves the overall survival in melanoma patients (Robert C, Karaszewska B, Schachter J et al. “Improved overall survival in melanoma with combined dabrafenib and trametinib. N Engl J Med 2015; 372:30-39), even though a substantial percentage of patients display intrinsic or acquired resistance to therapy (Manzano J L, Layos L, Buges C et al. “Resistant mechanisms to BRAF inhibitors in melanoma”. Ann Transl Med 2016; 4:237). Melanoma is extremely resistant to chemotherapeutic drugs, and apoptotic indices are typically low in melanomas, particularly at advanced stages (Glinsky G V, Glinsky V V, Ivanova A B and Hueser C J. “Apoptosis and metastasis: increased apoptosis resistance of metastatic cancer cells is associated with the profound deficiency of apoptosis execution mechanisms” Cancer Lett, 1997; 115: 185-193).

In general, the apoptotic machinery is altered in most human cancers, and treatment strategies often include the induction of cell death (Ghobrial I M, Witzig T E, Adjei A A. “Targeting apoptosis pathways in cancer therapy” CA Cancer J Clin, 2005; 55:178-94).

p75NTR (CD271) is a member of the tumour necrosis factor receptor superfamily with an extracellular domain that includes a ‘death’ domain similar to those present in other members of this family (He X L & Garcia K C (2004). Numerous studies have demonstrated a pro-apoptotic role of CD271 in the nervous system. Apoptosis via CD271 can be triggered by neurotrophins [nerve growth factor (NGF), brain-derived neurotrophic factor (BDNF), neurotrophin-3 (NT-3), and neurotrophin-4 (NT-4)], proneurotrophins and other ligands including aggregated amyloid and neurotoxic prion (Schor N F (2005) The p75 neurotrophin receptor in human development and disease. Prog. Neurobiol. 77: 201— 214). p75NTR can respond to NTs either independently or by modulating the affinity and specificity of the Trk receptors for NTs (He X, Garcia K C. “Structure of Nerve Growth Factor Complexed with shared Neurotrophin Receptor p75” Science, 2004; 304:870-875). While CD271 binds all NTs with equal low affinity, TrkA preferentially interacts with NGF, TrkB with BDNF and NT-4, and TrkC with NT-3 (Chao M V and Bothwell M, Neurotrophins: to cleave or not to cleave. Neuron. 2002; 33: 9-12). Amyloid-induced apoptosis in a neuroblastoma cell line is exclusively mediated by p75NTR (Frago L M, Leon Y, de la Rosa E J, Gomez-Munoz A and Varela-Nieto I (1998) Nerve growth factor and ceramides modulate cell death in the early developing inner ear. J. Cell Sci. 111:549-556).

Authors of the present invention have demonstrated that melanoma cells express and produce all of the NT and their receptors. All investigated melanoma cell lines express CD271 (Truzzi F, Marconi A, Lotti R, Dallaglio K, French L E, Hempstead B L, Pincelli C. “Neurotrophins and their receptors stimulate melanoma cell proliferation and migration” J Invest Dermatol, 2008; 128:2031-40). Melanoma cell lines also express the Trk high-affinity receptors. These tyrosine kinase receptors, that normally exert opposing effects to CD271, mediate migration and proliferation in melanoma.

Moreover, CD271 expression inversely correlates with hypoxia and melanoma invasiveness in vivo (Marconi A, Borroni R G, Truzzi F et al. “Hypoxia-Inducible Factor-1a and CD271 inversely correlate with melanoma invasiveness”. Exp Dermatol 2015; 24:396-398). In skin equivalent models, CD271 is highly expressed in early melanomas at the epidermal level and tends to disappear when melanoma starts to invade the dermis. In addition, CD271 is completely absent in skin reconstructs derived from metastatic cell lines. CD271 expression is highest in spheroids derived from primary melanoma cells, it decreases in cells derived from metastatic melanomas to disappear in highly invasive spheroids. CD271-negative cells are associated with a higher number of metastases in zebrafish, as compared with CD271-positive cells.

To treat CD271-related diseases, in particular melanoma, the need exists for molecules capable of selectively binding CD271.

### SUMMARY OF THE INVENTION

It has been surprisingly found that a peptide according to the invention, and more in particular the peptide Ac-DMet-DLeu-Gly-DIle-DIle-DAla-Gly-DLys-DAsn-DSer-Gly-DCys-NH₂ with a linear 20 kDa methoxy-PEG polymer conjugated to the D-Cys residue through the sulfur atom of the latter (herein defined as XYZ), is able to selectively bind the CD271 receptor. In particular, it was found that using retro-inverse sequences, in which the amino acids are in D configuration, instead of L, together with the total reversal of the sequence, provides a peptide that is able to bind the receptor without being even partially degraded by proteolytic enzymes, resulting in enhanced resistance to peptidase/protease. In order to render the peptide of the invention more stable, two types of carriers can be used: the first involves the conjugation of the peptide with polymer chains of PEG. Indeed, by PEGylation, renal filtration of the conjugated peptide is rather slow, if not completely eliminated. In addition, PEGylation allows to lengthen the residence in the circulation of vehicular and therapeutic peptides and proteins, preferentially in richly vascularized areas such as cancer. Secondly, it was found that liposomal systems not only render the peptide of the invention more stable, but also protect and mask the peptide. In addition, the surface of the liposomes can be engineered to direct the peptide specifically to the target.

The peptide of the invention induces apoptosis by activating the CD271 receptor. The peptide of the invention therefore allows reduction or growth arrest in cells of hyper-proliferative conditions and cancer, including melanoma.

### DETAILED DESCRIPTION OF THE INVENTION

It is therefore an object of the invention a pegylated peptide comprising or consisting of the amino acid sequence from the N-terminus to the C-terminus: R1-R2-Gly-R3-R4-R5-Gly-R6-R7-R8-Gly (SEQ ID NO:1) or the sequence from the N-terminus to the C-terminus: Gly-R8-R7-R6-Gly-R5-R4-R3-Gly-R2-R1 (SEQ ID NO:2) wherein:
R1 is selected from the group consisting of: Met, Ala, Val, Ile, Leu, Phe, Tyr and Trp
R2 is selected from the group consisting of: Leu, Met, Ala, Val, Ile and Phe
R3 is selected from the group consisting of: Ile, Met, Ala, Val, Leu and Phe
R4 is selected from the group consisting of: Ile, Met, Ala, Val, Leu and Phe
R5 is selected from the group consisting of: Ala, Met, Val, Ile, Leu and Phe
R6 is selected from the group consisting of: Lys and Arg
R7 is selected from the group consisting of: Asn, Gln, His, Ser, Thr, Tyr and Cys
R8 is selected from the group consisting of: Ser, Asn, Gln, His, Thr, Tyr and Cys
the N-terminus is W—CO—NH—, where W is a C₁-C₁₂ alkyl group or a hydrogen atom, or H₂N—
the C-terminus is —CO—N(Z₁)(Z₂), where Z₁ is an hydrogen atom or C₁-C₆ alkyl group and Z₂ is an hydrogen atom or C₁-C₆ alkyl group, or —COOH

and wherein R1 to R8 can be either in their D or L enantiomeric configuration

in which a linear or branched poly-(ethylene glycol) (PEG) polymer, preferably in the molecular weight (MW) range from 1 to 200 kDa, more preferably from 1 to 100 kDa is conjugated to the sequence or to the peptide, and wherein the non-conjugated end of PEG polymer is free or capped, preferably it is alkoxylated; and functional fragments or derivatives thereof.

Preferably the pegylated peptide according to the invention selectively binds the CD271 receptor.

Preferably said pegylated peptide further contains a D-Cys or L-Cys residue added at the C-terminus and/or at the N-terminus of the sequence. In a preferred embodiment the linear or branched PEG polymer is conjugated to either or both the N- and the C-terminus of the sequence; in a further preferred embodiment the linear or branched PEG polymer is conjugated to a Cys residue through the sulfur atom of the cystein-SH; even more preferably said PEG polymer is conjugated to the D-Cys or L-Cys residue added at the C-terminus and/or at the N-terminus of the sequence.

Preferably the pegylated peptide of the invention comprises or has the following sequence:

Ac-DMet-DLeu-Gly-DIle-DIle-DAla-Gly-DLys-DAsn-DSer-Gly-DCys-NH₂ (SEQ ID NO:5)

wherein the linear or branched PEG polymer conjugated to the D-Cys residue through the sulfur atom of the latter is a 20 kDa methoxy-PEG polymer.

Even more preferably the pegylated peptide is the peptide XYZ having the following structure:

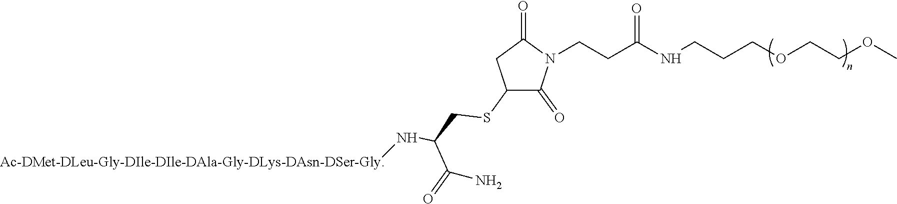

It is a further object of the invention a peptide having the following sequence:

DMet-DLeu-Gly-DIle-DIle-DAla-Gly-DLys-DAsn-DSer-Gly-DCys (SEQ ID NO:4)

wherein the N-terminus of the sequence is W—CO—NH—, where W is CH₃

and the C-terminus of the sequence is —CO—N(Z₁)(Z₂), where Z₁ and Z₂ are hydrogen atoms and functional fragments or derivatives thereof.

It is a further object of the invention a peptide comprising or consisting of the amino acid sequence from the N-terminus to the C-terminus:

(SEQ ID NO: 1)
R1-R2-Gly-R3-R4-R5-Gly-R6-R7-R8-Gly

or the sequence:

(SEQ ID NO: 2)
Gly-R8-R7-R6-Gly-R5-R4-R3-Gly-R2-R1

wherein:
R1 is selected from the group consisting of: Met, Ala, Val, Ile, Leu, Phe, Tyr and Trp
R2 is selected from the group consisting of: Leu, Met, Ala, Val, Ile and Phe
R3 is selected from the group consisting of: Ile, Met, Ala, Val, Leu and Phe
R4 is selected from the group consisting of: Ile, Met, Ala, Val, Leu and Phe
R5 is selected from the group consisting of: Ala, Met, Val, Ile, Leu and Phe
R6 is selected from the group consisting of: Lys and Arg
R7 is selected from the group consisting of: Asn, Gln, His, Ser, Thr, Tyr and Cys
R8 is selected from the group consisting of: Ser, Asn, Gln, His, Thr, Tyr and Cys
the N-terminus is W—CO—NH—, where W is a C₁-C₁₂ alkyl group or a hydrogen atom, or H₂N—
the C-terminus is —CO—N(Z₁)(Z₂), where Z₁ is an hydrogen atom or C₁-C₆ alkyl group and Z₂ is an hydrogen atom or C₁-C₆ alkyl group, or —COOH

and wherein R1 to R8 can be either in their D or L enantiomeric configuration

and functional fragments or derivatives thereof for use in the treatment of a CD271 related skin disease, preferably for use in the treatment of melanoma, Merkel-cell carcinoma, psoriasis, chronic skin ulcers and cutaneous squamous cell carcinoma, even more preferably for use in the treatment of melanoma.

Preferably said peptide selectively binds the CD271 receptor.

Preferably the above peptide comprises or has the following sequence:

(SEQ ID NO: 3)
DMet-DLeu-Gly-DIle-DIle-DAla-Gly-DLys-DAsn-DSer-
Gly

wherein the N-terminus of the sequence is W—CO—NH—, where W is CH₃

and the C-terminus of the sequence is —CO—N(Z₁)(Z₂), where Z₁ and Z₂ are hydrogen atoms and functional fragments or derivatives thereof.

In a preferred embodiment a D-Cys or L-Cys residue is added at the N- and/or at the C-terminus of the sequence or of the peptide of the invention.

In the peptide as above defined a linear or branched poly-(ethylene glycol) (PEG) polymer, preferably in the molecular weight (MW) range from 1 to 200 kDa, more preferably from 1 to 100 kDa, is conjugated to the sequence or to the peptide. The non-conjugated end of PEG polymer may be free or capped, preferably it is alkoxylated. Preferably said PEG is conjugated to a Cys residue through the sulfur cysteine-SH; still preferably said PEG polymer is conjugated to either the N- and/or the C-terminus of the sequence of the peptide of the invention

Another object of the invention is a bi- or multi-functional, linear or branched PEG polymer, preferably in the molecular weight (MW) range from 1 to 200 kDa, more preferably from 1 to 100 kDa, conjugated to at least two peptides as defined above or mixtures thereof

A further object of the invention is a lipid-based carrier or a liposome comprising a pegylated peptide or a peptide as defined above and at least one substance selected form the group consisting of a biodegradable and/or biocompatible lipid, cholesterol, a nanoparticle, a polymer or mixtures thereof.

As used herein, the term “lipid-based carrier” refers to a complex or structure having an internal environment separated from the external environment by a continuous layer of enveloping lipids. In the context of the present disclosure, the lipid-based carrier can mean a single layer lipid membrane (e.g. as found on a micelle or reverse micelle), a bilayer lipid membrane (e.g. as found on a liposome) or any multilayer membrane formed from single and/or bilayer lipid membranes. Therefore the term lipid-based carrier encompasses many different types of structures, including without limitation micelles, reverse micelles, unilamellar liposomes, multilamellar liposomes and multivesicular liposomes. In a preferred embodiment, peptides of the invention are comprised in a liposome structure,

A further object of the invention is a pharmaceutical composition comprising at least one pegylated peptide or at least one peptide or the bi- or multi-functional, linear or branched PEG or the liposome as defined above, and at least one pharmaceutically acceptable excipient and/or carrier, preferably in the form of an injectable pharmaceutical formulation.

It is an object of the invention the pegylated peptide, the peptide, the bi- or multi-functional, linear or branched PEG polymer, the liposome or the pharmaceutical composition as defined above for medical use, preferably for use in the treatment of CD271-related diseases or a CD271-related skin disease.

As used herein, CD271-related diseases include neuroblastoma, glioblastoma, astrocytoma, head and neck squamous cell carcinoma. More preferably, the CD271-related disease is a CD271-related skin diseases, therefore specifically including melanoma, psoriasis, Merkel-cell carcinoma, chronic skin ulcers, cutaneous squamous cell carcinoma. Even more preferably the CD271-related skin disease is melanoma.

It is an object of the invention the pegylated peptide, the peptide, the bi- or multi-functional, linear or branched PEG polymer, the liposome or the pharmaceutical composition as defined above, in combination with at least one chemotherapeutic agent and/or non-steroidal anti-inflammatory agent and/or immunotherapeutic agent and/or any treatment increasing the expression of CD271,

preferably the chemotherapeutic agent is selected from the group consisting of: dacarbazine, carmustine, cisplatin;

preferably the non-steroidal anti-inflammatory agent is selected from the group consisting of: ibuprofen, dexibuprofen, naproxen, fenoprofen, ketoprofen, dexketoprofen, flurbiprofen, oxaprozin, loxoprofen and mixtures thereof and/or mixtures of any treatments increasing the expression of CD271;

preferably the immunotherapeutic agent is selected from ipilimumab, nivolumab, pembrolizumab;

preferably the treatments increasing the expression of CD271 is selected from the BRAF/MET/KIT inhibitors including vemurafenib, trametinib, cobimetinib.

Preferably the chemotherapy agent is selected from the group consisting of: dacarbazine, carmustine, cisplatin and/or mixtures of any treatments increasing the expression of CD271.

Preferably the anti-inflammatory drugs (or agents) belong to the non-steroidal anti-inflammatory drugs (or agents) (NSAIDs) group. Preferably the non-steroidal anti-inflammatory agent is selected from the group consisting of: ibuprofen, dexibuprofen, naproxen, fenoprofen, ketoprofen, dexketoprofen, flurbiprofen, oxaprozin, loxoprofen and mixtures thereof and/or mixtures of any treatments increasing the expression of CD271.

The invention further provides acombination comprising:
a. at least one pegylated peptide, or at least one peptide or the bi- or multi-functional, linear or branched PEG polymer or the liposome as above defined; and
b. at least one chemotherapeutic agent and/or non-steroidal anti-inflammatory agent, and/or immunotherapeutic agent and/or any treatment increasing the expression of CD271,
   preferably the chemotherapeutic agent is selected from the group consisting of: dacarbazine, carmustine, cisplatin;
   preferably the non-steroidal anti-inflammatory agent is selected from the group consisting of: ibuprofen, dexibuprofen, naproxen, fenoprofen, ketoprofen, dexketoprofen, flurbiprofen, oxaprozin, loxoprofen and mixtures thereof and/or mixtures of any treatments increasing the expression of CD271;
   preferably the immunotherapeutic agent is selected from ipilimumab, nivolumab, pembrolizumab;
   preferably the treatments increasing the expression of CD271 is selected from the BRAF/MET/KIT inhibitors including vemurafenib, trametinib, cobimetinib.

It is a further object of the invention a pharmaceutical composition comprising the above combination and at least one pharmaceutically acceptable excipient and/or carrier, preferably in the form of an injectable pharmaceutical formulation; preferably said combination is for use in the treatment of a CD271-related disease or for use in the treatment of a CD271-related skin disease.

Further objects of the invention are a nucleic acid sequence encoding the peptide as above defined, an expression vector comprising said nucleic acid sequence or a host cell comprising said expression vector.

The present invention further provides a method for activating CD271 comprising contacting CD271 with the peptide is the peptide or the derivative as defined above or the lipid-based carrier comprising at least one peptide as defined above or the bi- or multi-functional, linear or branched PEG polymer as defined above or the liposome.

Other objects of the invention are an antibody or fragment thereof that binds to the peptide according to the invention and a peptide multimer comprising at least one peptide according to the invention.

The present invention includes PEGylated derivative of the peptides according to the invention.

A further object of the invention is the peptide or the derivative as defined above or the lipid-based carrier comprising at least one peptide as defined above or the bi- or multi-functional, linear or branched PEG polymer as defined above or the liposome as defined above for use in the reduction or in the growth arrest of primary and metastatic melanoma.

In the general formula reported above chemical groups, forming amide bonds, may be added to the N-terminal amino group or to the C-terminal carbonyl of the peptide or of the sequence.

According to the invention, the N-terminus of the present peptides or of the sequences as above defined may be either free (i.e. H₂N—) or acylated (i.e. W—CO—NH—, where W is a hydrogen atom or a C₁-C₁₂ alkyl group)

The C-terminus of the present peptides or of the sequences as above defined may be either free (i.e. —COOH) or amidated C-terminus (i.e. —CO—N(Z₁)(Z₂), where Z₁ is an hydrogen atom or C₁-C₆ alkyl group and Z₂ is an hydrogen atom or C₁-C₆ alkyl group).

Further to the above, the C-terminus and/or the N-terminus of the present peptides or of the sequences as above defined may be linked to a PEG polymer. The PEG polymer can be linked to the free terminal carboxy or to the free terminal amino groups of the present peptides through appropriate chemical derivatization.

In the peptides according to the invention all amino acids can be either in their L or D enantiomeric configuration, with the obvious exception of Gly.

In a preferred embodiment, the present invention provides a peptide comprising or having the sequence: Ac-DMet-DLeu-Gly-DIle-DIle-DAla-Gly-DLys-DAsn-DSer-Gly-DCys-NH₂ (SEQ ID NO:4) and functional fragments or derivatives thereof.

In a more preferred embodiment, in the above peptide the DCys residues is conjugated to a linear or branched PEG polymer, preferably in the molecular weight (MW) range from 1 to 200 kDa, more preferably from 1 to 100 kDa, even more preferably 20 KDa. Preferably, the non-conjugated end of PEG polymer is free or capped, preferably it is alkoxylated. Preferably the PEG polymer is a linear 20 kDa methoxy-PEG polymer conjugated to the peptide through the sulfur atom of the D-Cys residue.

Preferably, when the PEG polymer or the linear 20 kDa methoxy-PEG polymer is conjugated to the Cys residue of the peptide or of the sequence, said conjugation occurs by reacting the cysteine-SH as indicated below:

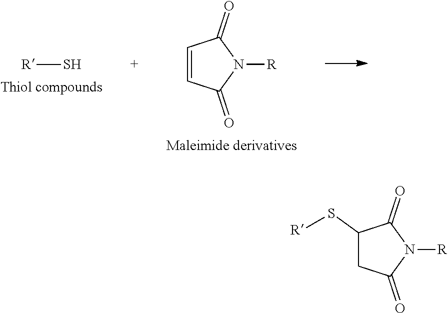

wherein the maleimide derivatives have the following structure:

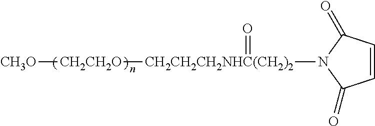

If a hydrogen is added to the N-terminus a free amine at the N-terminus is obtained; similarly, an acetyl group can be added thus generating an N-terminal acetylated peptide. If a hydroxyl group is added a terminal carboxylic acid is generated. If an amine group is added to the carbonyl acid terminal of the oligopeptide sequence or of the peptide an amide is formed.

In the context of the present invention, for Ac it is intended that the terminus of the sequence is W—CO—NH—, and W is CH₃.

Ac is preferably at the N-terminus of the sequence or of the peptide.

In the context of the present invention, for NH₂ it is intended that the terminus of the sequence is —CO—N(Z₁)(Z₂), and Z₁ and Z₂ are hydrogen atoms.

NH₂ is preferably at the C-terminus of the sequence or of the peptide.

“Polyethylene glycol” or “PEG” refers to polymers of ethylene glycol, in a branched or straight chain, represented by the general formula HO(CH₂CH₂O)ₙH, where n is an integer of at least 2. PEG includes polymers of ethylene glycol with an average total molecular weight selected from the range of about 500 to about 50,000 Daltons or more. The average molecular weight of a PEG chain is indicated by a number, e.g., PEG-5,000 or Peg-5K refers to polyethylene glycol chain having a total molecular weight average of about 5,000 Daltons, or about 5 kiloDaltons or kDa or K (1 kDa=1K=1000 Daltons). Various sizes of PEG are commercially available and may be used in the products and methods described herein, e.g., having an average size of about 1 to 200, preferably 1 to 100 kDa. In an embodiment, the PEG moieties described herein have an average molecular weight of less than or equal to about 20 kDA

PEGylation (i.e., attachment of a PEG moiety) of the peptides described herein may be carried out by methods known in the art. For example, a reactive PEGylation reagent that is reactive to a specific amino acid side chain may be used. An example is a thiol- or sulfhydryl-reactive PEG, e.g., having a maleimide moiety (PEG-maleimide), which can react with the Cys side chain. A further example is an amine-reactive PEG, e.g., having an N-hydroxysuccinimide ester (PEG-NHS ester), which can react with an amine side chain (e.g., Lys). In embodiments, PEG modification may be at the N- and/or C-terminus, using reagents which are amine- or carboxy-reactive. In further embodiments, PEG modification may be through a linker, i.e., a linker or spacer moiety located between the PEG moiety and the peptide. Such linkers include, for example, heterobifunctional or homobifunctional reagents. PEG derivatives containing aldehyde activation groups can be specifically linked to the N-terminus of peptides by reductive alkylation. PEG derivatives can also be linked to the C-terminus of peptides through for example coupling reagents.

The term “functional derivative” is used herein to denote a chemical derivative of the presently described peptides having the same physiological function as the corresponding unmodified counterpart or, alternatively, having the same in vitro function in a functional assay (for example, in one of the assays described in one of the examples disclosed herein).

A pharmaceutical composition is formulated to be compatible with its intended route of administration. Examples of routes of administration include parenteral, e.g., intravenous, intradermal, subcutaneous, oral, inhalation, transdermal (topical), transmucosal, and rectal administration.

The invention relates also to a polynucleotide coding for the peptides as above defined, to a vector comprising the above polynucleotide and to a host cell genetically engineered which expresses the peptide as above defined. Preferably the polynucleotide is selected from the group consisting of: RNA or DNA, preferably said polynucleotide is DNA.

Preferably the vector is an expression vector selected from the group consisting of: plasmids, viral particles and phages.

Preferably said host cell is selected from the group consisting of: bacterial cell, fungal cell, insect cell, animal cell and plant cell, preferably said host cell is an animal cell.

The peptides of the invention are in the form of synthetic or recombinant, linear and multimeric peptides in any chemical, physical and/or biological form such as to maintain their function.

The peptides of the invention may be synthetized and used in the branched form as Multiple Antigenic Peptide (MAP), as disclosed e.g. in the patent U.S. Pat. No. 5,229,490.

All the amino acids in the peptide may have the same stereochemistry, for example, the peptide may consist of only L-amino acids or only D-amino acids. Alternatively, the peptide may comprise a combination of both L- and D-amino acids.

The peptide of the present invention may be in the form of a dimer or multimer. In the present specification, examples of spacers comprised in the dimer or multimer include ester bonds (—CO—O—, —O—CO—), ether bonds (—O—), amide bonds (NHCO, CONH), sugar chain linkers, polyethylene glycol linkers, peptide linkers, and the like. Examples of peptide linkers include linkers containing at least one of 20 natural amino acids that constitute a protein. The number of amino acids of the peptide linker is, for example, but not limited to, 1 to 20, 1 to 15, 1 to 12, 1 to 10, 1 to 8, 1 to 6, or 1 to 4. Examples of peptide linkers include arginine dimer, arginine trimer, arginine tetramer, lysine dimer, lysine trimer, lysine tetramer, glycine dimer, glycine trimer, glycine tetramer, glycine pentamer, glycine hexamer, alanine-alanine-tyrosine (AAY), isoleucine-leucine-alanine (ILA), arginine-valine-lysine-arginine (RVKR), and the like. The spacer may be divalent or multivalent.

When the peptide of the present invention is a multimer, a branched multivalent linker (e.g., dendrimer), a metal complex, or the like may be used for linkage.

Examples of the branched multivalent linker include diethylenetriamine, spermine, spermidine, triethanolamine, ethylenediaminetetraacetate (EDTA), pentaerythritol, azido-propyl(alkyl)amine, lysine, ornithine, aspartic acid, glutamic acid, polyfunctional peptides (lysine, ornithine, or asparagic acid- or glutamic acid-containing dipeptide, tripeptide, or tetrapeptide), and organic multivalent amino compounds (e.g., poly(amidoamine) (PAMAM), tris(ethyleneamine)ammonia, and poly(propyleneimine) (Astramol (trademark))). The dendrimer of the present invention includes, for example, a dimer obtained by connecting the N-terminals (or C-terminals) of peptides herein disclosed. Further, the dendrimer of the present invention also includes a tetramer obtained by further connecting a “dimer obtained by connecting the N-terminals of the above peptides” and a “dimer obtained by connecting the C-terminals of the above peptides.”

Included in the present invention are also derivates or variants of the peptides above defined. Suitably, “derivatives” or “variants” include those in which instead of the naturally occurring amino acid the amino acid which appears in the sequence is a structural analog thereof. Amino acids used in the sequences may also be derivatized or modified, e.g. labelled, providing the function of the peptide is not significantly adversely affected. Derivatives and variants as described above may be prepared during synthesis of the peptide or by post-production modification, or when the peptide is in recombinant form using the known techniques of site-directed mutagenesis, random mutagenesis, or enzymatic cleavage and/or ligation of nucleic acids.

Functionally “fragments” according to the invention may be made by truncation, e.g. by removal of one or more amino acids from the N and/or C terminal ends. Such fragments may be derived from the sequences herein disclosed or may be derived from a functionally equivalent peptide as described above.

Suitably, functional variants or derivatives according to the invention have an amino acid sequence which has more than 70%, e.g. 75 or 80%, preferably more than 85%, e.g. more than 90 or 95% homology to the sequences herein disclosed.

Peptides of the invention, as defined herein, may be chemically modified, for example, post-translationally modified. For example, they may be glycosylated or comprise modified amino acid residues. They can be in a variety of forms of polypeptide derivatives, including amides and conjugates with polypeptides.

Chemically modified peptides also include those having one or more residues chemically derivatized by reaction of a functional side group. Such derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzyl histidine.

Also included as chemically modified peptides are cyclised peptides, i.e. peptides of the invention which are linked with a covalent bond to generate a ring. Typically an amino terminus and a carboxy terminus (so called head-to-tail cyclisation), an amino terminus and a sidechain (so called head-to-sidechain cyclisation), carboxy terminus and a sidechain (so called sidechain-to-tail cyclisation), or a side chain and a side chain (so called sidechain-to-sidechain cyclisation) may be linked with a covalent bond to form a cyclic peptide. Head-to-tail cyclic peptides may typically be formed by amide bond formation. Sidechain-to-sidechain cycles may typically be formed via Cys-Cys disulfide bridge formation or amide bond formation within a cyclic peptide. Alternatively, an amino terminus, a carboxy terminus or a side chain may be linked with a covalent bond to the peptide backbone to form a cyclic peptide.

Also included as chemically modified peptides are those which contain one or more naturally occurring amino acid derivatives of the twenty standard amino acids. For example, 4-hydroxyproline may be substituted for proline or homoserine may be substituted for serine.

A peptide of the invention may carry a revealing label. Suitable labels include radioisotopes, fluorescent labels, enzyme labels, or other protein labels such as biotin.

Any formula given herein is also intended to represent unlabeled forms as well as isotopically labeled forms of the peptides. Isotopically labeled peptides have structures depicted by the formulas given herein except that one or more atoms are replaced by an atom having a selected atomic mass or mass number. Examples of isotopes that can be incorporated into peptides of the invention include isotopes of hydrogen, carbon, nitrogen, oxygen, phosphorous, fluorine, and chlorine. Pharmaceutically acceptable solvates in accordance with the invention include those wherein the solvent of crystallization may be isotopically substituted, e.g. D2O, d6-acetone, d6-DMSO.

Peptides as described above for use in accordance with the invention may be prepared by conventional modes of synthesis including genetic or chemical means.

Synthetic techniques, such as a solid-phase Merrifield-type synthesis, may be preferred for reasons of purity, antigenic specificity, freedom from unwanted side products and ease of production. Suitable techniques for solid-phase peptide synthesis are well known to those skilled in the art (see for example, Merrifield et al., 1969, Adv. Enzymol 32, 221-96 and Fields et al., 1990, Int. J. Peptide Protein Res, 35, 161-214). Chemical synthesis may be performed by methods well known in the art involving cyclic sets of reactions of selective deprotection of the functional groups of a terminal amino acid and coupling of selectively protected amino acid residues, followed finally by complete deprotection of all functional groups. Synthesis may be performed in solution or on a solid support using suitable solid phases known in the art.

In an alternative embodiment a peptide of the invention may be produced from or delivered in the form of a polynucleotide which encodes, and is capable of expressing, it. Such polynucleotides can be synthesised according to methods well known in the art, as described by way of example in Sambrook et al (1989, Molecular Cloning—a laboratory manual; Cold Spring Harbor Press). Such polynucleotides may be used in vitro or in vivo in the production of a peptide of the invention. Such polynucleotides may therefore be administered or used in the treatment of cancer or another disease or condition as described herein.

The present invention also includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al (ibid).

Thus, the peptide may be provided by delivering such a vector to a cell and allowing transcription from the vector to occur. Suitably, a polynucleotide of the invention or for use in the invention in a vector is operably linked to a control sequence which is capable of providing for the expression of the coding sequence by the host cell, i.e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be for example, plasmid, virus or phage vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

The invention also includes cells that have been modified to express a peptide of the invention. Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors encoding for a peptide of the invention include mammalian HEK293T, CHO, HeLa and COS cells. Suitably the cell line selected will be one which is not only stable, but also allows for mature glycosylation and cell surface expression of a polypeptide. Expression may be achieved in transformed oocytes. A suitable peptide may be expressed in cells of a transgenic non-human animal, in particular a mouse. A transgenic non-human animal expressing a peptide of the invention is included within the scope of the invention. A peptide of the invention may also be expressed in Xenopus laevis oocytes or melanophores.

The present invention also extends to antibodies (monoclonal or polyclonal) and their antigen-binding fragments (e.g. F(ab)2, Fab and Fv fragments i.e. fragments of the “variable” region of the antibody, which comprises the antigen binding site) directed to peptides as defined hereinbefore, i.e. which bind to epitopes present on the peptides and thus bind selectively and specifically to such peptides, and which may be used in the methods of the invention.

The peptides of the present invention may be employed alone as a sole therapy or in combination with other therapeutic agents for the prevention and/or treatment of the above-mentioned conditions.

Also provided are compositions including one or more peptides or polynucleotides described herein. Such compositions typically include a pharmaceutically acceptable carrier. As used herein “pharmaceutically acceptable carrier” includes, but is not limited to, saline, solvents, dispersion media, coatings, antibacterial and antifungal agents, isotonic and absorption delaying agents, and the like, compatible with pharmaceutical administration. Additional compounds can also be incorporated into the compositions.

A composition may be prepared by methods known in the art of pharmacy. In general, a composition can be formulated to be compatible with its intended route of administration. A formulation may be solid or liquid. Administration may be systemic or local. In some aspects local administration may have advantages for site-specific, targeted disease management. Local therapies may provide high, clinically effective concentrations directly to the treatment site, with less likelihood of causing systemic side effects.

Examples of routes of administration include parenteral (e.g., intravenous, intradermal, subcutaneous, intraperitoneal, intramuscular), enteral (e.g., oral or rectal), and topical (e.g., epicutaneous, inhalational, transmucosal) administration. Appropriate dosage forms for enteral administration of the compound of the present invention may include tablets, capsules or liquids. Appropriate dosage forms for parenteral administration may include intravenous administration. Appropriate dosage forms for topical administration may include nasal sprays, metered dose inhalers, dry-powder inhalers or by nebulization. Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in, for instance, ampoules, disposable syringes or multiple dose vials made of glass or plastic.

Compositions can include sterile aqueous solutions or dispersions and sterile powders for the extemporaneous preparation of sterile solutions or dispersions. For intravenous administration, suitable carriers include human albumin, physiological saline, bacteriostatic water, Cremophor EL™ (BASF, Parsippany, N.J.) or phosphate buffered saline. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, albumin, water, ethanol, polyol (for example, glycerol, propylene glycol, and liquid polyethylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin. Sterile solutions can be prepared by incorporating the active compound (e.g., a peptide or polynucleotide described herein) in the required amount in an appropriate solvent with one or a combination of ingredients such as those enumerated above, as required, followed by filtered sterilization. Generally, dispersions are prepared by incorporating the active compound into a sterile vehicle, which contains a dispersion medium and other ingredient such as from those enumerated above. In the case of sterile powders for the preparation of sterile injectable solutions, methods of preparation that may be used include vacuum drying and freeze-drying which yields a powder of the active ingredient plus any additional desired ingredient from a previously sterile-filtered solution thereof.

For enteral administration, a composition may be delivered by, for instance, nasogastric tube, enema, colonoscopy, or orally. Oral compositions may include an inert diluent or an edible carrier. For the purpose of oral therapeutic administration, the active compound can be incorporated with excipients and used in the form of tablets, troches, or capsules. Oral compositions can also be prepared using a fluid carrier. Pharmaceutically compatible binding agents can be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds may be delivered in the form of an aerosol spray, a nebulizer, or an inhaler, such as a nasal spray, metered dose inhaler, or dry-powder inhaler. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds may be formulated into ointments, salves, gels, or creams as generally known in the art. An example of transdermal administration includes iontophoretic delivery to the dermis or to other relevant tissues. The active compounds can also be prepared in the form of suppositories (e.g., with conventional suppository bases such as cocoa butter and other glycerides) or retention enemas for rectal delivery. The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially. Liposomal suspensions can also be used as pharmaceutically acceptable carriers. These can be prepared according to methods known to those skilled in the art. Delivery reagents such as lipids, cationic lipids, phospholipids, liposomes, and microencapsulation may also be used.

As is common practice, the compositions are normally accompanied by written or printed instructions for use in the treatment in question.

The expert in the art will select the form of administration and effective dosages by selecting suitable diluents, adjuvants and/or excipients.

The peptide can be administered as separate compositions (simultaneous, sequential) of the individual components of the treatment or as a single dosage form containing both agents. When the peptide of this invention are in combination with others active ingredients, the active ingredients may be separately formulated into single-ingredient preparations of one of the above-described forms and then provided as combined preparations, which are given at the same time or different times, or may be formulated together into a two—or more—ingredient preparation.

The peptides as above defined may be administered to a patient in a total daily dose of, for example, from 0.1 to 500 mg/kg body weight daily. Dosage unit compositions may contain such amounts of submultiples thereof to make up the daily dose. The determination of optimum dosages for a particular patient is well known to one skilled in the art.

The present invention is therefore illustrated by means of non-limiting examples in reference to the following figures.

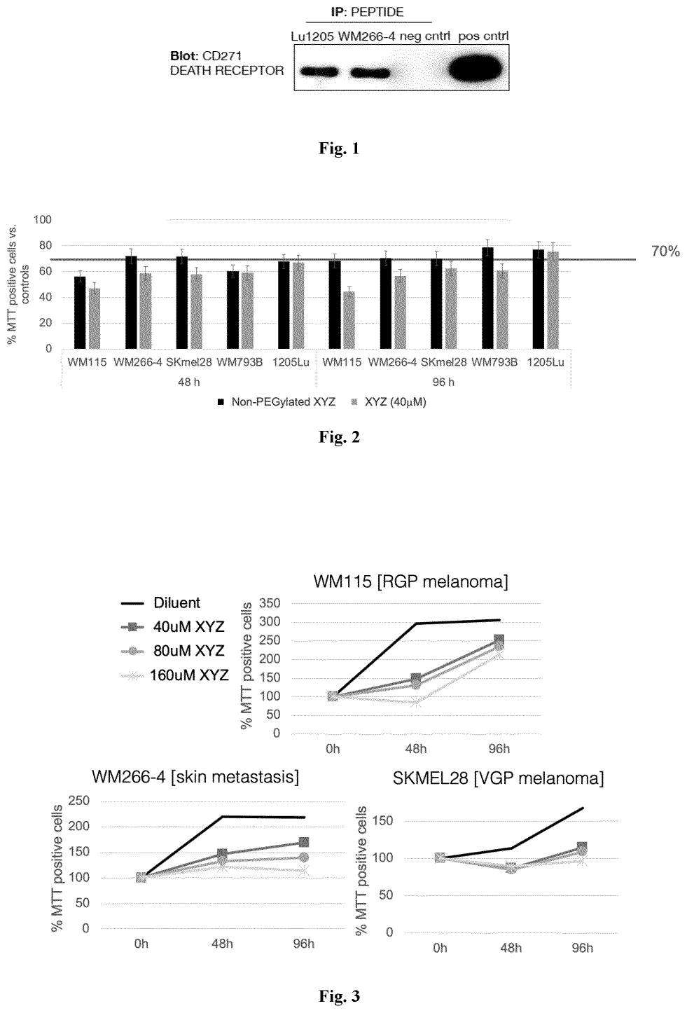

FIG. 1. The peptide immunoprecipitate with CD271 in two metastatic melanoma cell lines. Protein extracts from two melanoma cell lines were immunoprecipitated with the Pegylated form of the peptide (XYZ) and blotted against CD271. The sepharose beads-protein lysates (without CD271) is used as negative control, while no-immunoprecipitated protein lysate from WM266-4 is used as positive control.

FIG. 2. The peptide or the NON-PEGylated form reduce melanoma cell proliferation. Five primary or metastatic melanoma cell lines were cultured and treated with either the PEGylated and non-PEGylated form of the peptide (Ac-DMet-DLeu-Gly-DIle-DIle-DAla-Gly-DLys-DAsn-DSer-GlyNH2) and evaluated by MTT assay at 48 and 96 hrs.

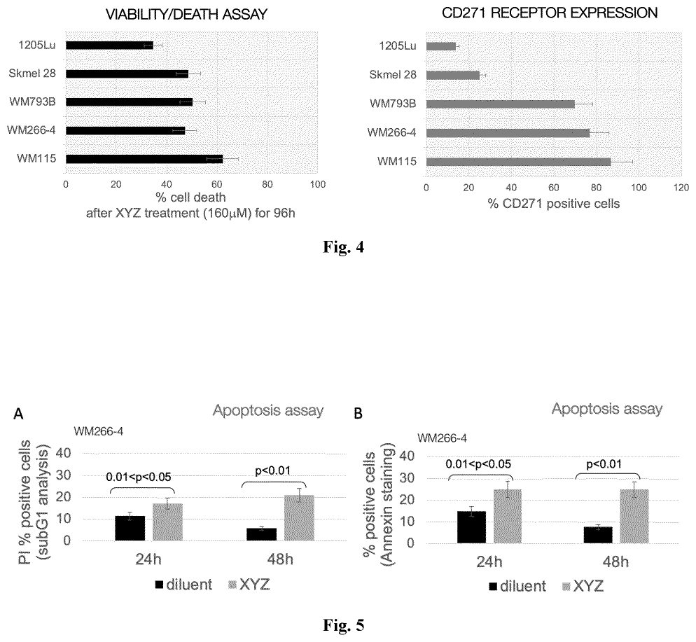

FIG. 3. The peptide reduces cell proliferation in primary and metastatic melanoma cell lines. Three primary or metastatic melanoma cell lines were cultured, treated with the peptide (XYZ) at different doses and evaluated by MTT assay at different time points

FIG. 4. The peptide-induced cell death correlates with CD271 expression levels in melanoma cell lines. Five primary and metastatic melanoma cell lines were cultured and viability measured by Trypan blue staining solution. Flow cytometry was used to evaluate the expression level of CD271 receptor.

FIG. 5. The peptide significantly increases apoptosis in melanoma cells. WM266-4 melanoma cell line was cultured and the rate of apoptosis measured after treatment with the peptide (XYZ) as compared to diluent at different time points by flow cytometry (A) counting % of cells in sub-G1 with propidium iodide staining or (B) counting % of Annexin positive cells staining. Student's t-test was used for comparison of the means

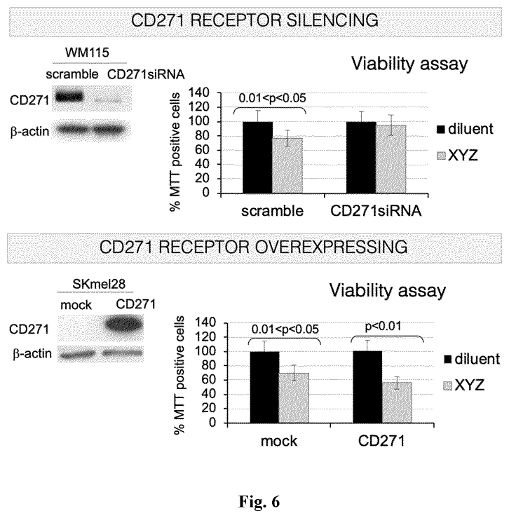

FIG. 6. The peptide reduces melanoma cell viability through CD271. WM115 melanoma cell line was transiently transfected with CD271 siRNA, as shwon by western blotting. MTT assay was used to measure cell viability after treatment with the peptide (XYZ) or the diluent in siRNA and scramble cells. SKme128 melanoma cell line was transiently transfected with CD271 mRNA, as shown by western blotting. MTT assay was used to measure cell viability after treatment with the peptide (XYZ) or the diluent in siRNA and scramble cells. Student's t-test was used for comparison of the means.

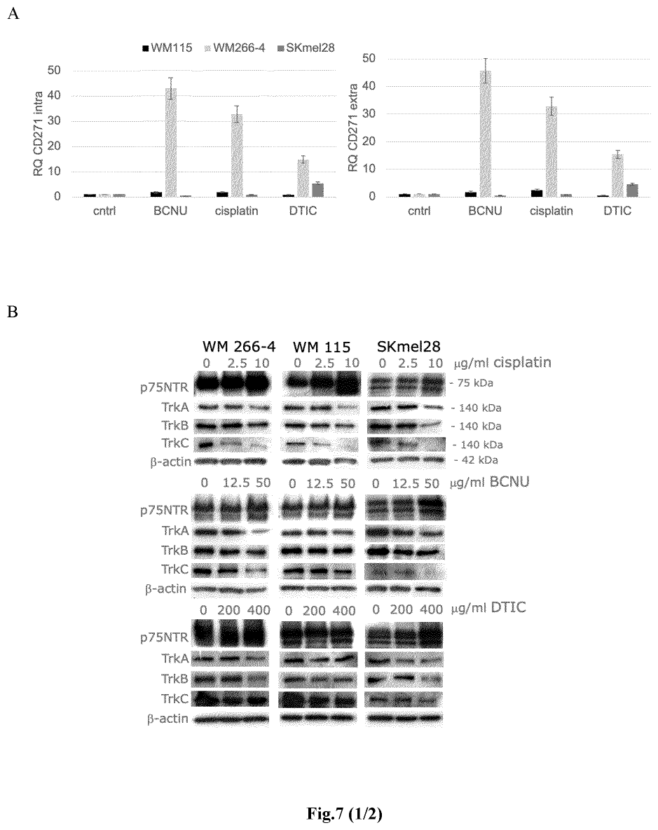

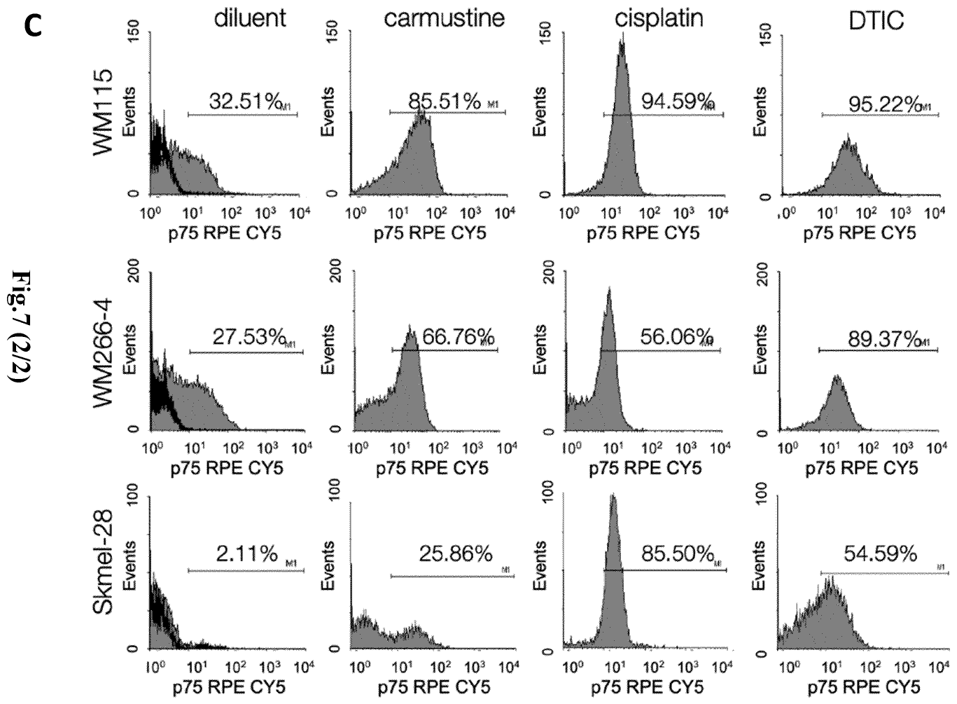

FIG. 7. Chemotherapy up-regulates CD271 in primary and metastatic melanoma cell lines. (A) A real-time PCR using primers for intra- and extracellular CD271 receptor was performed on RNA extracts from three melanoma cell lines after treatment with carmustine (BCNU), cisplatin, dacarbazine (DTIC) or diluent. (B) Protein extracts from the three melanoma cell lines treated with different doses of cisplatin, carmustine, dacarbazine or diluent were separated on polyacrylamide gel and transferred onto nitrocellulose membranes. (C) Melanoma cell lines were treated with the chemotherapy or the diluent (see above) stained with anti CD271 mAb and analysed by flow cytometry.

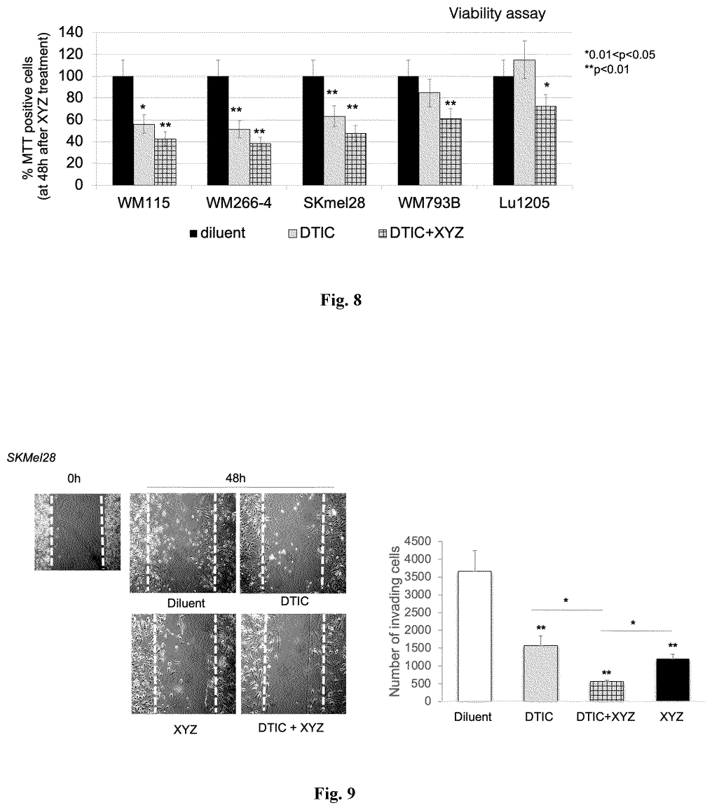

FIG. 8. The peptide augments dacarbazine (DTIC)-induced reduction of melanoma cell proliferation. Five melanoma cell lines were treated with DTIC, DTIC in combination with the peptide (XYZ) or with diluent alone. Proliferation was measured by MTT assay. Student's t-test was used for comparison of the means.

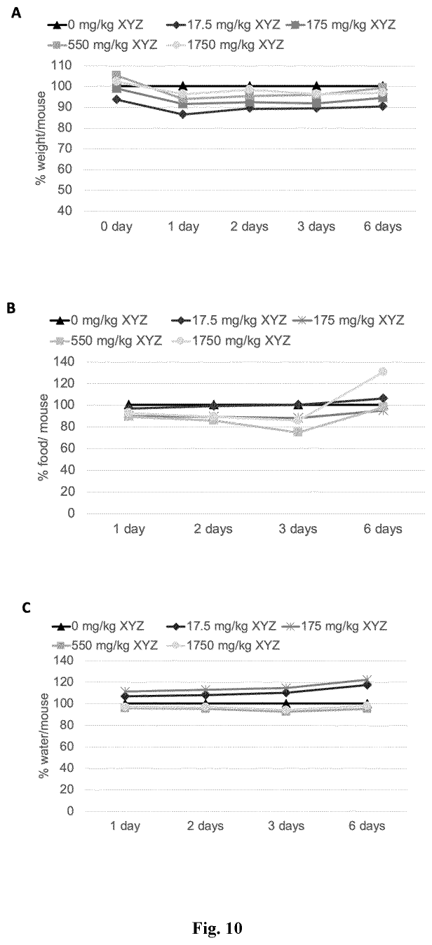

FIG. 9. The peptide augments dacarbazine-induced melanoma cell migration. Melanoma cell line SKMel28 plated onto 24-well Boyden/Matrigel chambers was stimulated with the peptide, the peptide in combination with DTIC or the diluent alone. Number of invading cells was measured at 48 hrs by the scratch-wound assay. Student's t-test was used for comparison of the means

FIG. 10. No toxic effects in 6 mice treated with XYZ. Acute toxicity testing was performed in CD1 mice with different doses of XYZ up to six days after treatment. After one day of fasting, six mice were injected intravenously (28 G needle) through the tail vein with a total dose of 17.5, 175, 550 or 1750 mg/kg/mouse of XYZ in a physiological solution. 50 gr of food were available two hours later. The health status (coat and appearance) of mice was observed from the first day after treatment. (a) Mice were weighed prior to XYZ injection and then every day until the sixth day. The amount of ingested food (b) and drinking water (c) were measured.

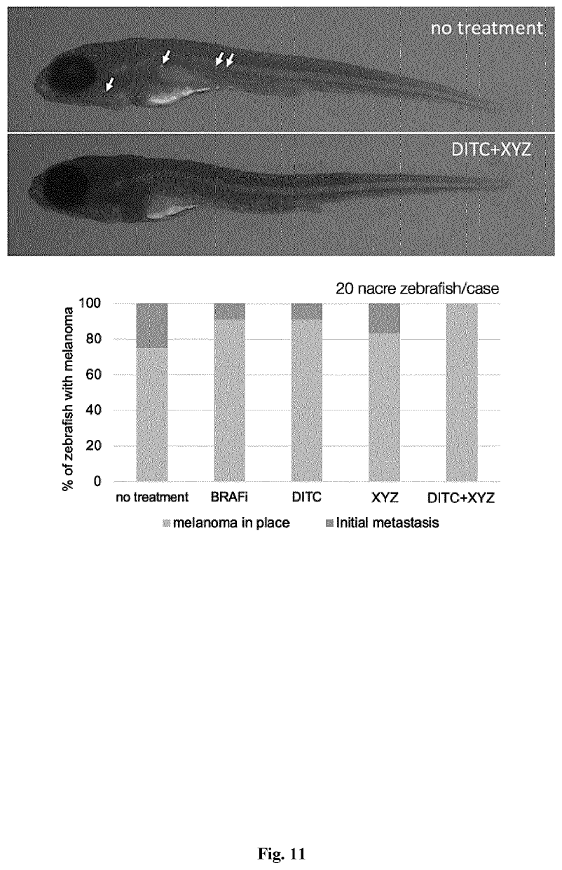

FIG. 11. The peptide reduces initial metastasis formation in zebrafish injected with melanoma cells. Metastatic melanoma cells from a patient with BRAF mutation were treated with the peptide, DTIC, the peptide in combination with DTIC, DTIC alone, BRAF inhibitor or left untreated. They were stained with Vibrant Cell Labelling Solution (red) and injected into the yolk of transparent zebrafish larvae and monitored. The number of embryos with metastasis was counted at 5 days post injection.

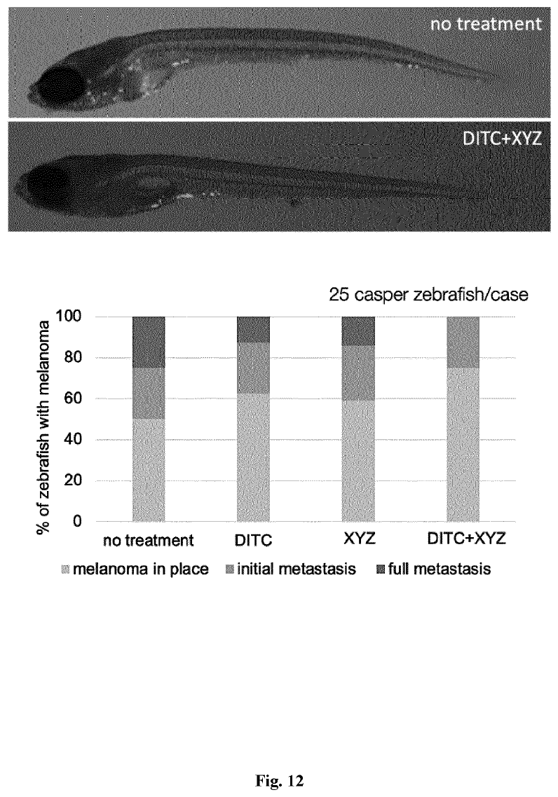

FIG. 12. The peptide, in combination with DTIC, blocks the formation of full metastasis. SKMel28 melanoma cell line was treated with XYZ, DTIC, the peptide in combination with DTIC, or left untreated. They were stained with Vibrant Cell Labelling Solution (red) and injected into the yolk of transparent zebrafish larvae and monitored. The number of embryos with metastasis was counted at 5 days post injection.

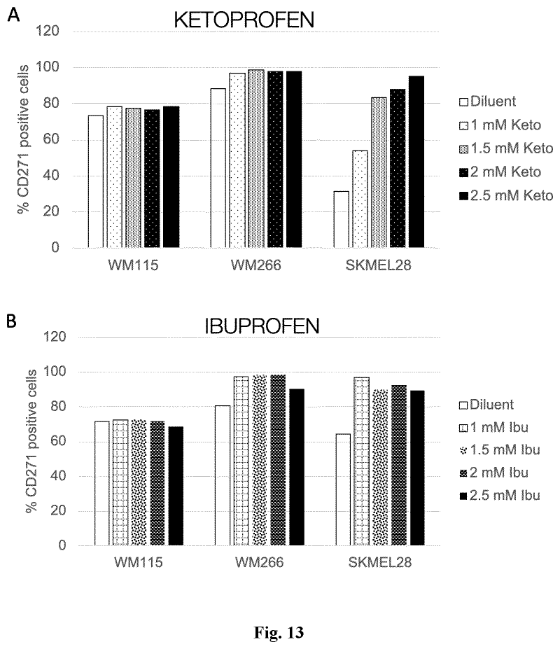

FIG. 13. Non-steroid anti-inflammatory drugs (NSAID) enhance CD271 levels in melanoma cell lines. Three melanoma cell lines were treated with increasing doses of ketoprofen (A) or ibuprofen (B) and the percentage of CD271 positive cells was measured by flow cytometry.

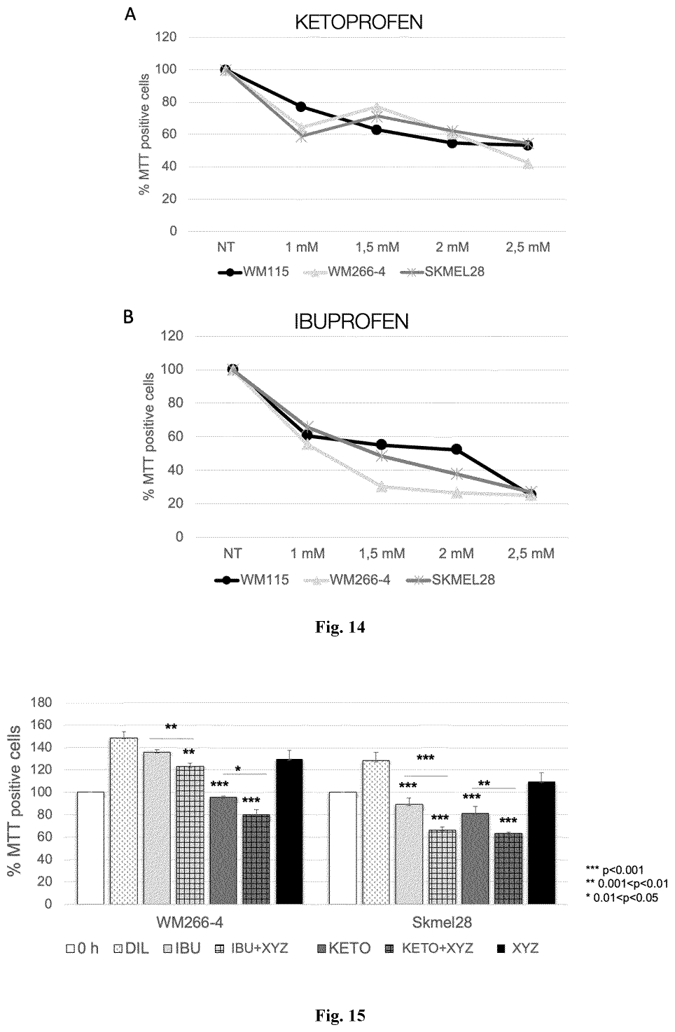

FIG. 14. NSAID reduce melanoma cell line proliferation. Three melanoma cell lines were treated with increasing concentrations of either ketoprofene (A) or ibuprofene (B). Proliferation was measured by MTT assay at 48 hrs.

FIG. 15 The peptide, in combination with NSAID, further reduces melanoma cell line proliferation Two melanoma cell lines were treated with the peptide alone, ibuprofene or ketoprofene alone or in combination with the peptide. Proliferation was measured by MTT assay at 48 hrs. Student's t-test was used for comparison of the means

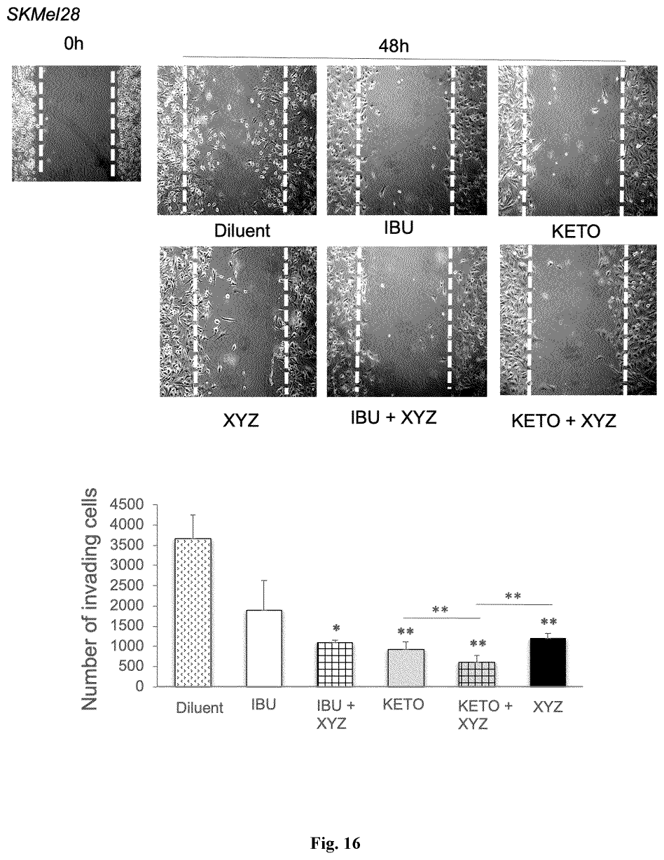

FIG. 16. The combination of the peptide with NSAID decreases cell migration in melanoma cell lines. SKMel 28 melanoma cell line was treated with the peptide alone, ibuprofene or ketoprofene alone or in combination with the peptide. Number of invading cells was measured at 48 hrs by the scratch-wound assay. Student's t-test was used for comparison of the means. * 0.01<p<0.05; ** p<0.01

### EXAMPLES

### Materials and Methods

### Peptide synthesis and PEGylation

The synthesis of the different peptides was performed at NeoMPS/PolyPeptide Laboratories (Strasbourg, France) using Solid-Phase Peptide Synthesis with standard tBoc chemistry and preparative Reversed-Phase Chromatography purification.

Peptide indicated as XYZ corresponds to the following structure:

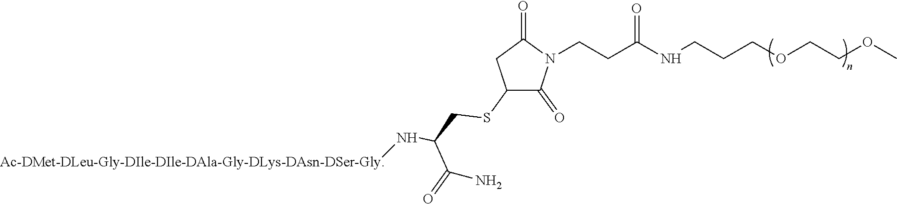

The PEGylation of the DCys-containing peptide to give XYZ was performed as per the following protocol.

Materials:

Ac-dM-12-dC-NH₂ peptide, MW 1204Da (NeoMPS/PolyPeptide laboratories, p/n SP081269 lot n AW11015); mPEG20 kDa-maleimide (MW 21721Da: NOF Co., SUNBRIGHT ME-200MA lot M60519); Sodium acetate buffer 20 mM pH 5.0; Ammonium acetate 20 mM

Procedure:

In a 50 ml polypropylene Falcon tube with magnetic stirrer, 10.6 mg (8.3×10⁻³ mmol) of peptide are dissolved in 10 ml of acetate buffer at room temperature. Then, 162.4 mg (7.47×10⁻³ mmol, 0.9 eq) of PEG are added. After 2 h the HPLC (see analytical method) shows complete conversion of mPEG. The buffer of the reaction mixture is then exchanged to 20 mM ammonium acetate (see buffer exchange method) and lyophilized. Purification of the resulting solid by RP-HPLC (see preparative HPLC method) with further lyophilization of the fractions of interest yields 99 mg of conjugate.

Analytical Method:

Column: Phenomenex Jupiter C18, 5 μm, 300 Å, 4.6×250 mm

Flow: 1 ml/min

Eluents: A) water 0.1% trifluoroacetic acid (vol/vol)
B) acetonitrile 0.1% trifluoroacetic acid (vol/vol)

Detector: UV 214 and 280 nm, ELSD (mPEG-maleimide is detectable at UV)

Elution: 5 min at A/B 90/10
Gradient to 45/55 in 45 min
Wash step at 100% B for 7 min
Re-equilibration at 90/10 for 7 min

Retention times: peptide ˜29.5 min
peptide dimer ˜34.0 min
mPEG-maleimide ˜36.2 min
XYZ ˜36.8 min

Buffer Exchange Method:

Column: GE Healthcare HiPrep 26/10 Desalting

Flow: 10 ml/min

Eluant: Ammonium acetate 20 mM

Preparative HPLC Method:

Column: Phenomenex Jupiter C18, 5 μm, 300 Å, 15×250 mm

Flow: 10 ml/min

Sample injection: 1 ml of 50 mg/ml solution in eluent A/ eluent B 90/10

Eluents and elution program as per the analytical method.

### Melanoma Cell Lines and Primary Human Cells

WM-115 (Part of the Wistar Special Collection, Catalog N. CRL-1675), WM-266-4 (Part of the Wistar Special Collection, Catalog N. CRL-1676), and SK-MEL-28 (Catalog N. HTB-72) melanoma cell lines (American Type Culture Collection, Manassas, Va., USA) were maintained in BME Melanoma Medium composed by: BME (Lonza, Basel, Switzerland) supplemented with 10% Fetal Bovine Serum (FBS), 2 mM L-glutamine, 1 mM sodium pyruvate, 0.1 mM non-essential amino acids, 1% penicillin/streptomycin and 1.5 g/L sodium bicarbonate. WM793-B (Part of the Wistar Special Collection, Catalog N. CRL-2806), and 1205Lu (Part of the Wistar Special Collection, Catalog N. CRL-2812), melanoma cell lines (American Type Culture Collection, Manassas, Va., USA) were cultured in Tumor Medium 2% containing: MCDB154CF medium (Thermo Fisher Scientific Inc, Waltham, Mass., USA) supplemented with 2% FBS, 1.5 g/L sodium bicarbonate, Leibovitz L-15 medium, 2 mM L-glutamine, 200 mM CaCl2, 5 mg/ml insulin and 1%. penicillin/streptomycin (Lonza, Basel, Switzerland).

The use of melanoma biopsies was approved by the ethical committee (Comitato Etico dell'Area Vasta Emilia Nord). Biopsies were taken from the University Hospital Policlinico of Modena and patient gave inform consent. Immediately after surgical resection, melanoma cells were dissociated into single cells with collagenase (1 mg/ml; Biochem, Nuoro, Italy) as previously indicated (Civenni et al., 2011). The single cell suspension was filtered and single cells were harvested and seeded as spheroids according to the liquid overlay method. Cells were maintained in RPMI, 10% decomplemented FBS, 2 mM L-glutamine and 1% penicillin/streptomycin and used to perform Zebrafish xenotransplant.

### Immunoprecipitation

1205Lu and WM266-4 melanoma cell lines were treated with or without XYZ. 24 h later cells were harvested with RIPA buffer (10 mM Tris, pH 8.0; 150 mM NaCl; 1% Nonidet P-40, 0.5% deoxycholate; 0.1% SDS) containing protease inhibitors. Nuclei were removed by centrifugation (11,000 rpm for 3 min). Monoclonal antibody anti-p75NTR (Upstate) was bound to sepharose beads lh at 4° C. under rotation. Then, pre-cleared lysates were conjugated to sepharose beads-antibody complex or to sepharose beads alone as negative control, overnight at 4° C. under rotation. Immunocomplexes were washed with Port Buffer (10 mM Tris-HCl, pH 8.5; 150 mM NaCl; 0.1% Renex 30; 0.01% BSA; 2.5% NaN3) six times and with PT Buffer (10 mM Tris-HCl, pH 8.5; 150 mM NaCl; 0.5% Tween 20; 2.5% NaN3) three times. Samples were eluted with 1× Laemnli sample buffer for 5° C. at 90° C. and Western Blotting for p75NTR was performed.

### MTT Assay

5×103 cells/well were seeded in 96-well culture plates. At different time points, cells were incubated with 0.5% MTT (3-(4,5-dimethylthiazol-2-yl)-2,5-diphenyltetrazolium bromide) for 4 h at 37° C. and then dissolved with 100 μl Isopropanol with 0.04N HCl. The plate was read at 560 nm with a reference filter of 650 nm. Results are expressed as viability percentage, as compared to control.

### CD271 Expression by Flow Cytometry Analysis

Melanoma cell lines were treated with the chemotherapy or the diluent for 48 h, then were harvested and incubated with anti CD271 antibody (1:100 in PBS, Lab Vision Corporation, Thermo Fisher Scientific, Fremont, Calif., USA) for 20 min at 4° C. Cells were labeled with secondary antibody Alexa Fluor anti-mouse 488 (1:50, Thermo Fisher Scientific) for 20 min at 4° C. and analyzed with Epics XL flow cytometer (Beckman Coulter).

### Cell Death Analysis by Flow Cytometry

WM266-4 cell line was treated with the peptide (XYZ) or diluent at different time points. After 24 and 48 h, cells were trypsinised and resuspended in hypotonic fluorochrome solution: 50 μg/ml propidium iodide containing 0.1% sodium citrate and 0.5% Triton X-100 (Sigma Aldrich, Milano, Italy). After 15 min, cells were analyzed using an Epics XL flow cytometer (Coulter Electronics Inc., Hialeah, Fla., USA). Apoptosis was detected by evaluating the reduced fluorescence of the DNA-binding dye PI in the apoptotic nuclei.

### Annexin V Staining

WM266-4 cell line was treated with the peptide (XYZ) or diluent at different time points. After 24 and 48 h, cells were trypsinised, collected by centrifugation and resuspended in 500 μL of binding buffer containing annexin V-FITC and propidium iodide (PI). After incubation at room temperature for 5 min in the dark, annexin V-FITC binding was analyzed by flow cytometry using FITC signal detector and PI staining by the phycoerythrin emission signal detector.

### Melanoma Cells Transfection with siRNA

Melanoma cells were plated in 2D for 24 h in antibiotic free medium. WM115 cell line was then transfected with scrambled or CD271 siRNA (50 nM and 100 nM, respectively) (Dharmacon Inc, Lafayette, Colo., USA) in antibiotic/FBS free medium supplemented with 0.1% BSA and 48 h later cells were seeded for MTT assay or lysed for western blotting.

### Melanoma Cells Infection

Skme128 cell line was plated according to the liquid overlay method. After 24 h, cells were transduced by infection with viral supernatant generated by CD271-LNSN packaging cells or by LNSN packaging cells (kindly provided from F. Mavilio) in appropriate medium for melanoma, as described previously in the presence of polybrene (8 μg/ml). 48 h after infection cells were lysed for WB analysis or seeded for MTT assay.

### Real Time PCR

Total cellular RNA was extracted from the three melanoma cell lines after chemotherapy treatment using TRI Reagent method performed according to manufacturer instructions (Sigma-Aldrich). Quantitative Real time PCR was performed with an ABI 7500 (Applied Biosystems, Foster City, Calif., USA) for sortilin. As an internal control, housekeeping gene beta-actin mRNA expression was measured in separated tube. RNAse-free H2O was used as a negative control. lmicrog of RNA was subjected to retro-transcription and amplification in a 50 μl reaction mixture using the One-Step RT-PCR Master Mix Reagents kit (Applied Biosystems). Both sortilin and beta-actin real time PCR were performed using Pre-Developed TaqMan Assay Reagents (CD271 MGB probe was FAM dye labelled; beta-actin MGB probe was VIC dye labelled, Applied Biosystems). Thermal cycling conditions for One-Step RT-PCR were: initial reverse transcription at 48° C. for 30 min, then DNA Polymerase activation at 95° C. for 10 min, followed by 40 cycles of denaturation at 95° C. for 15 s, annealing/extension at 60° C. for 1 min. Data from each sample were compared with CD271 expression of no-treated cells, as calibrators, using the Sequence Detection Software Version 1.2.3 according to the Relative Quantification (ddCt) Study method (Applied Biosystems). Results were obtained as the mean from three independent experiments. Double sided Student's t-test was performed between samples and calibrator.

### Western Blotting

Melanoma spheroids were disaggregated and harvested for CD271 in lysis buffer pH 7.5 (150 mM NaCl, 15 mMMgCl, 1 mM EGTA, 50 mM Hepes, 10% Glycerol, 1% Triton). Membranes were first incubated in blocking buffer and then overnight at 4° C. with primary antibodies: anti-human CD271 mouse monoclonal antibody (1:1000, Upstate, Lake Placid, NY, USA),), anti-human TrkB rabbit polyclonal antibody (1:750, Upstate), anti-human TrkC goat polyclonal antibody (1:750, Upstate), anti-human TrkA rabbit polyclonal antibody (1:1000, Upstate) and anti-human beta-actin monoclonal antibody (1:5000, Sigma-Aldrich). Membranes were then incubated for 45 min at room temperature with the following peroxidase-conjugated secondary antibodies: goat anti-mouse (1:3000, Biorad, Hercules, Calif., USA) for p75NTR and beta-actin; goat anti-rabbit (1:3000, Biorad) for TrkA and TrkB; donkey anti-goat (1:1000, Santa Cruz Biotechnology Inc Santa Cruz, Calif.) for TrkC. Membranes were washed and developed using the ECL chemiluminescent detection system (Amersham Biosciences UK Limited, Little Chalfont Buckinghamshire, England). The band intensity was quantitatively determined using ImageJ software (Wayne Rasband).

### Scratch-Wound Assay

50,000 cells were plated on six-well tissue culture plates and were treated with 10 microg/ml mitomycin C for 2 hours. 24 hours later, cells were washed three times in serum-free medium and three lines for each well were drawn along the cell monolayer with a sterile plastic tip. Plates were washed twice with serum-free medium to remove all detached cells and incubated in serum-free medium with XYZ, chemotherapy, ibuprofen or ketoprofen alone or in combination with the peptide. Cells were monitored at 48 hours from stimulation. The result of each experiment was expressed as the mean of migrated cells from six different areas. The final results are expressed as the mean±SD of three different experiments. Student's T-test was used for comparison of the means.

### Zebrafish Handling for Xeno-Transplantation

Zebrafish handling was performed at the Zebrafish Centre of the University of Padova, Italy, under ethical committee (OPBA) authorization 407/2015-PR. Zebrafish embryos were obtained from natural spawning of albino adults, raised following standard protocols (Westerfield, M et al., 2000) and staged according to Kimmel et al. (1995). For xeno-transplantation, embryos were mechanically dechorionated at 2 days post-fertilization (dpf), anesthetized with tricaine 0.16 mg/ml and placed along plastic lanes immersed in 2% methylcellulose/PBS. Melanoma human cells were stained with Vybrant Cell-Labeling Solution (5 ug/ml, Molecular Probes) or CFSE (2 uM, Invitrogen) for 20 minutes at 37° C. according to the manufacturer. Stained cells were loaded in a glass capillary needle and microinjected into the yolk (about 50 cells/embryo), using a WPI PicoPump apparatus. Xenotransplanted embryos were grown at 33° C., treated with was treated with XYZ, DTIC, the peptide in combination with DTIC, or left untreated. Zebrafishes were monitored daily and documented from 1 day post injection (dpi) up to 1 week (experimental endpoint). Imaging was performed using a Leica MZFLIII dissecting microscope equipped with a Leica DFC7000T camera. Panels were assembled using Adobe Photoshop CC v. 14.0×64.

### RESULTS

To demonstrate that the peptide is able to directly bind CD271, total protein lysates of WM266-4 and Lu1205 melanoma cell lines were immunoprecipitated with the peptide conjugated to sepharose beads. Immunocomplexes were washed with Port Buffer and with PT Buffer, then eluted with Laemnli sample buffer for 5′ at 90° C. The samples were run onto 7% polyacrylamide gel, transferred onto nitrocellulose membrane and blotted with anti-CD271 monoclonal antibody. The sepharose beads-peptide complex without protein lysates is used as negative control, while total protein lysate from WM266-4 without sepharose beads-peptide complex is used as positive control (FIG. 1).

Five melanoma cell lines were used for testing the efficacy of XYZ or its non-PEGylated peptide component: WM115 and WM266-4, which are respectively the primary and metastatic melanoma derived from the same patient and expressing high levels of CD271, and SKme128, a metastatic melanoma cell line expressing low levels of CD271, WM793B, a primary melanoma, and 1205Lu derived from lung metastatic melanoma. The viability of the cell culture is assessed by MTT conversion. MTT solution is applied to the plates and placed into a cell culture incubator for 4 hours. The blue formazan metabolite produced by viable cells is then extracted into isopropanol and absorbance is measured at 570 nm using a spectrophotometer. XYZ or the non-PEGylated peptide reduce cell proliferation, as shown by MTT assay, in all melanoma cells 48 h and 96 h after treatment, with no significant difference (FIG. 2), indicating that PEG conjugation does not alter peptide efficacy.

In addition, XYZ diminishes cell proliferation, as shown by MTT assay, in a dose-dependent manner in WM115, WM266-4 and SKme128 cell lines (FIG. 3).

Because CD271 mediates apoptosis in many cell systems, we analysed the effect of XYZ in melanoma cell lines in relation to the levels of the receptor. After treatment, melanoma cell lines were harvested and incubated with Trypan Blue staining solution or incubated with CD271 antibody for flow cytometer analysis. XYZ is more efficacious in cell lines expressing higher levels of CD271, as shown by flow cytometry analysis and under a microscope viable cell counting (FIG. 4).

In particular, XYZ triggers melanoma apoptosis, a most well-known type of programmed cell death in culture. Indeed, XYZ induces a significantly higher rate of apoptosis than diluent alone in two apoptosis assays (Propidium iodide, subG1 analysis; Annexin staining) (FIG. 5). In details, melanoma cell lines, gently were trypsinised and washed once with serum-containing media before incubation with annexin V-FITC or with propidium iodide. 24 h and 48 h later, FITC signal detector or sub G0/G1 peak for annexin or PI staining respectively, were observed by flow cytometry.

Moreover, XYZ triggers melanoma cell death in culture through CD271. XYZ fails to reduce cell viability (MTT assay) in the WM115 CD271 silenced (siRNA) melanoma cell line, while it induces cell death to a greater extent in SKMe128 melanoma cell lines overexpressing CD271 (CD271 viral vector) (FIG. 6). This indicates that CD271 mediates XYZ effects in melanoma.

It was also found that chemotherapeutic agents dose-dependently upregulate CD271 expression at the mRNA (Real-time PCR) and protein level (Western blotting and flow cytometry) (FIG. 7). Therefore, total cellular RNA was extracted from melanoma cell lines using TRI Reagent method.

To verify the quality of the RNA, we ran an agarose minigel in RNase-free conditions. 1 ml of RNA was subjected to retro-transcription and amplification in a 50 μl reaction mixture using the One-Step RT-PCR Master Mix Reagents kit. Quantitative Real Time PCR for CD271 receptor was performed by an ABI 7500 Real-Time PCR System. Regarding protein expression, melanoma cells were harvested in lysis buffer pH 7.5, run onto 7% polyacrylamide gel and transferred onto nitrocellulose membrane. Membranes were first incubated in blocking buffer and with primary CD271 antibody, then with secondary antibody. Finally, membranes were washed and developed using the ECL chemiluminescent detection system.

Indeed, XYZ (180 mM) combined with DTIC (200 mg/ml) is significantly more effective in reducing cell proliferation (MTT assay) than the chemotherapeutic agent alone 48 h after treatment (FIG. 8). Moreover, XYZ combined with DTIC, inhibits melanoma cell migration significantly more than either DTIC or XYZ alone as shown by scratch-wound assay with SKmel28 cell line (FIG. 9).

XYZ did not show any acute toxic effect (appearance, weight, water and food intake), as shown by the up and down procedure. Intravenous injections were performed in six CD1 mice (Charles River, Calco (MI) Italy) tails for administration from 17.5 mg/kg to 1750 mg/kg XYZ (FIG. 10).

Zebrafish model represents an alternative xenotransplant model in vivo that offers a rapid and efficient approach for assessing drug effects in human cancer cells. (Haldi M, Ton C, Seng W L, McGrath P: Human melanoma cells transplanted into zebrafish proliferate, migrate, produce melanin, form masses and stimulate angiogenesis in zebrafish. Angiogenesis 2006; 9:139-151). Zebrafish embryos are particularly useful for microscopic analysis as they are translucent, thus offering the opportunity to visualize the metastatic process at high resolution (Stoletov K, Montel V, Lester R D, Gonias S L, Klemke R: High-resolution imaging of the dynamic tumour cell vascular interface in transparent zebrafish; PNAS 2007; 104:17406-17411). We have recently shown that CD271 down-regulation promotes melanoma progression and metastasis in zebrafish (Saltari A, Truzzi F, Quadri M, Lotti R, Palazzo E, Grisendi G, Tiso N, Marconi A, Pincelli C: CD271 down-regulation promotes melanoma progression and invasion in three-dimensional models and in zebrafish; J Invest Dermatol 2016; 136:2049-2058).

For xenotransplantation, albino embryos were mechanically dechorionated at 2 days post-fertilisation, anesthetized with tricaine 0.16 mg/ml and placed along plastic lanes immersed in 2% methylcellulose/PBS. SKmel28 cells or melanoma cells from patient metastasis were stained with Vybrant Cell-Labeling Solution. Stained cells were loaded in a glass capillary needle and microinjected into the yolk (about 50 cells/embryo), Xenotransplanted embryos were monitored daily and documented from one day post injection up to one week.

XYZ alone reduces metastasis formation in zebrafish injected with melanoma cells derived from patient with BRAF mutation, as compared to control. Moreover, XYZ, in combination with DITC, further enhances this effect (FIG. 11).

Moreover, in xenotransplanted zebrafish with SKmel 28 cell line from primary melanoma DTIC or XYZ reduces full metastasis, while XYZ in combination with DTIC blocks the formation of full metastasis (FIG. 12).

There is growing evidence that inflammation may exacerbate cancer metastasis and several clinical studies show that taking nonsteroidal anti-inflammatory drugs (NSAIDs) appears to reduce metastases. TNF-alpha upregulates malignant melanoma migration in vitro and this could be reduced by ibuprofen both in solution and delivered from a hydrogel (Redpath M, Marques C M, Dibden C, Waddon A, Lalla R, Macneil S: Ibuprofen and hydrogel-released ibuprofen in the reduction of inflammation-induced migration in melanoma cells; Br J Dermatol. 2009; 161:25-33).

Khwaja et al. observed that at higher concentrations, some NSAIDs inhibit proliferation and induce apoptosis of cancer cells. They also observed that p75NTR is an important upstream modulator of the anticancer effects of NSAIDs and that ibuprofen induction of the p75NTR protein establishes an alternate mechanism by which ibuprofen may exert an anticancer effect (Khwaja F 1, Allen J, Lynch J, Andrews P, Djakiew D: Ibuprofen inhibits survival of bladder cancer cells by induced expression of the p75NTR tumor suppressor protein; Cancer Res. 2004; 64:6207-13)

Epidemiologic studies show that patients chronically consuming NSAIDs for arthritis exhibit a reduced incidence of prostate cancer. In addition, some NSAIDs show anticancer activity in vitro. NSAIDs exert their anti-inflammatory effects by inhibiting cyclooxygenase (COX) activity; however, evidence suggests that COX-independent mechanisms mediate decreased prostate cancer cell survival. R-flurbiprofen and ibuprofen have been found to selectively induce p75NTR-dependent decreased survival of prostate cancer cells independently of COX inhibition (Quann E J, Khwaj a F, Zavitz K H, Djakiew D: The aryl propionic acid R-flurbiprofen selectively induces p75NTR-dependent decreased survival of prostate tumor cells; Cancer Res. 2007; 67:3254-62).

Ketoprofen is a widely used NSAID that also exhibits cytotoxic activity against various cancers. A carboranyl analogue prodrug ester of ketoprofen exhibited high cytostatic activity against melanoma and colon cancer cell lines, with the most pronounced activity was found in cell lines that are sensitive to oxidative stress (Buzharevski A, Paskas S, Laube M, Lönnecke P, Neumann W, Murganic B, Mijatovic S, Maksimovic-Ivanic D, Pietzsch J, Hey-Hawkins E: Carboranyl Analogues of Ketoprofen with Cytostatic Activity against Human Melanoma and Colon Cancer Cell Lines; ACS Omega. 2019; 4:8824-8833).

We found that 2D melanoma cell cultures (WM115, WM266-4 and SKMEL28) treated with ketoprofen or ibuprofen from 1 to 2.5 mM show increased CD271 protein levels after 48 h treatment (FIG. 13).

We also demonstrated that treatment with ketoprofen and ibuprofen dose-dependently decreases proliferation in CD271-positive melanoma cell cultures (WM115, WM266-4 and SKMEL28) (FIG. 14).

In our experiments, two different 2D melanoma cell cultures (WM266-4 and SKMEL28), treated with a combination of XYZ with either ibuprofen or ketoprofen, clearly showed a statistically significant decrease in cell survival (i.e. increased efficacy) when compared to the treatment with the anti-inflammatory agent alone (MTT assay), suggesting a synergistic effect between the intrinsic cytotoxic effect of the anti-inflammatory agent and the targeting of CD271 by XYZ (FIG. 15).

The combination of XYZ with anti-inflammatory treatments such as ibuprofen and ketoprofen was also shown to decrease cell migration in 2D melanoma cell cultures, as shown by scratch-wound assay with SKme128 cell line (FIG. 16).

1. A pegylated peptide comprising the amino acid sequence from the N-terminus to the C-terminus:
(SEQ ID NO: 1)
R1-R2-Gly-R3-R4-R5-Gly-R6-R7-R8-Gly
or of the amino acid sequence from the N-terminus to the C-terminus:
(SEQ ID NO: 2)
Gly-R8-R7-R6-Gly-R5-R4-R3-Gly-R2-R1
wherein:
R1 is selected from the group consisting of: Met, Ala, Val, Ile, Leu, Phe, Tyr and Trp;
R2 is selected from the group consisting of: Leu, Met, Ala, Val, Ile and Phe;
R3 is selected from the group consisting of: Ile, Met, Ala, Val, Leu and Phe;
R4 is selected from the group consisting of: Ile, Met, Ala, Val, Leu and Phe;
R5 is selected from the group consisting of: Ala, Met, Val, Ile, Leu and Phe;
R6 is selected from the group consisting of: Lys and Arg;
R7 is selected from the group consisting of: Asn, Gln, His, Ser, Thr, Tyr and Cys;
R8 is selected from the group consisting of: Ser, Asn, Gln, His, Thr, Tyr and Cys;
the N-terminus is W—CO—NH—, where W is a C₁-C₁₂ alkyl group or a hydrogen atom, or H₂N—;
the C-terminus is —CO—N(Z₁)(Z₂), where Z₁ is an hydrogen atom or C₁-C₆ alkyl group and Z₂ is an hydrogen atom or C₁-C₆ alkyl group, or —COOH;
and wherein R1 to R8 can be either in their D or L enantiomeric configuration;
in which a linear or branched poly-(ethylene glycol) (PEG) polymer is conjugated to at least one of the amino acids of the peptide, and wherein the non-conjugated end of PEG polymer is free or capped.

2. The pegylated peptide according to claim 1 further comprising a D-Cys or L-Cys residue linked to the C-terminus and/or at the N-terminus thereof.

3. The pegylated peptide according to claim 1, wherein the linear or branched PEG polymer is conjugated to either or both the N- and the C-terminus of the peptide.

4. The pegylated peptide according to claim 1, wherein the linear or branched PEG polymer is conjugated to a Cys residue through the sulfur atom of the latter.

5. The pegylated peptide according to claim 4 being Ac-DMet-DLeu-Gly-DIle-DIle-DAla-Gly-DLys-DAsn-DSer-Gly-DCys-NH₂ (SEQ ID NO:5)
wherein the linear or branched PEG polymer conjugated to the D-Cys residue through the sulfur atom of the latter is a 20 kDa methoxy-PEG polymer.

6. The pegylated peptide according to claim 5 having the following structure (XYZ):

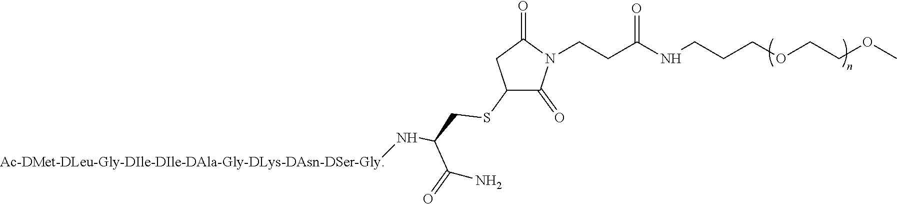

7. A peptide according to claim 1, having the following amino acid sequence from the N-terminus to the C-terminus:
DMet-DLeu-Gly-DIle-DIle-DAla-Gly-DLys-DAsn-DSer-Gly-DCys (SEQ ID NO:4)
wherein the N-terminus of the sequence is W—CO—NH—, where W is CH₃
and the C-terminus of the sequence is —CO—N(Z₁)(Z₂), where Z₁ and Z₂ are hydrogen atoms and functional fragments or derivatives thereof.

8. A bi- or multi-functional, linear or branched PEG polymer, conjugated to at least two peptides according to claim 1.

9. A liposome comprising at least one peptide according to claim 1 and at least one substance selected from the group consisting of a biodegradable and/or biocompatible lipid, cholesterol, a nanoparticle, a polymer or mixtures thereof.

10. A pharmaceutical composition comprising a peptide according to claim 1 and at least one pharmaceutically acceptable excipient and/or carrier, optionally in the form of an injectable pharmaceutical formulation.

11. A method for the treatment of CD271-related diseases, comprising administering a peptide of claim 1 to a patient in need thereof.

12. The method of claim 11, wherein the CD271-related diseases are selected from neuroblastoma, glioblastoma, astrocytoma, head and neck squamous cell carcinoma, psoriasis, Merkel-cell carcinoma, chronic skin ulcers, cutaneous squamous cell carcinoma and melanoma.

13. The pharmaceutical composition of claim 10 further comprising a chemotherapeutic agent and/or non-steroidal anti-inflammatory agent and/or immunotherapeutic agent and/or any treatment increasing the expression of CD271, wherein
the chemotherapeutic agent is optionally selected from the group consisting of: dacarbazine, carmustine, cisplatin;
the non-steroidal anti-inflammatory agent is optionally selected from the group consisting of: ibuprofen, dexibuprofen, naproxen, fenoprofen, ketoprofen, dexketoprofen, flurbiprofen, oxaprozin, loxoprofen and mixtures thereof;
the immunotherapeutic agent is optionally selected from ipilimumab, nivolumab, pembrolizumab; and
the treatments increasing the expression of CD271 are optionally selected from the BRAF/MET/KIT inhibitors including vemurafenib, trametinib, cobimetinib.

14. A method for the treatment of a CD271 related skin disease, comprising administering a peptide comprising the amino acid sequence from the N-terminus to the C-terminus:
(SEQ ID NO: 1)
R1-R2-Gly-R3-R4-R5-Gly-R6-R7-R8-Gly
or the sequence from the N-terminus to the C-terminus:
(SEQ ID NO: 2)
Gly-R8-R7-R6-Gly-R5-R4-R3-Gly-R2-R1
wherein:
R1 is selected from the group consisting of: Met, Ala, Val, Ile, Leu, Phe, Tyr and Trp
R2 is selected from the group consisting of: Leu, Met, Ala, Val, Ile and Phe
R3 is selected from the group consisting of: Ile, Met, Ala, Val, Leu and Phe
R4 is selected from the group consisting of: Ile, Met, Ala, Val, Leu and Phe
R5 is selected from the group consisting of: Ala, Met, Val, Ile, Leu and Phe
R6 is selected from the group consisting of: Lys and Arg
R7 is selected from the group consisting of: Asn, Gln, His, Ser, Thr, Tyr and Cys
R8 is selected from the group consisting of: Ser, Asn, Gln, His, Thr, Tyr and Cys
the N-terminus of the sequence or of the peptide is W—CO—NH—, where W is a C₁-C₁₂ alkyl group or a hydrogen atom, or H2N—
the C-terminus of the sequence or of the peptide is —CO—N(Z₁)(Z₂), where Z₁ is an hydrogen atom or C₁-C₆ alkyl group and Z₂ is an hydrogen atom or C₁-C₆ alkyl group, or —COOH
and wherein R1 to R8 can be either in their D or L enantiomeric configuration
and functional fragments or derivatives thereof to a patient in need thereof.

15. The method according to claim 14 wherein the CD271 related skin disease is selected from melanoma, Merkel-cell carcinoma, psoriasis, chronic skin ulcers and cutaneous squamous cell carcinoma.

16. The method according to claim 15 wherein the CD271 related skin disease is melanoma.

17. The method according to claim 14, wherein the peptide comprises sequence:
(SEQ ID NO: 3)
DMet-DLeu-Gly-DIle-DIle-DAla-Gly-DLys-DAsn-DSer-
Gly
wherein the N-terminus of the sequence or of the peptide is W—CO—NH—, where W is CH₃ and the C-terminus of the sequence or of the peptide is —CO—N(Z₁)(Z₂), where Z₁ and Z₂ are hydrogen atoms
or functional fragments or derivatives thereof.

18. The method of claim 14, wherein a D-Cys or L-Cys residue is added at the C-terminus and/or at the N-terminus of the sequence.

19. The method according to claim 14, wherein a linear or branched poly-(ethylene glycol) (PEG) polymer is conjugated to at least one of the amino acids of the peptide,
wherein the non-conjugated end of PEG polymer is optionally free or capped, preferably it is alkoxylated.

20. (canceled)

21. (canceled)

22. The method according to claim 14, further comprising administering at least one chemotherapeutic agent and/or non-steroidal anti-inflammatory agent and/or immunotherapeutic agent and/or immunotherapeutic agent and/or any treatment increasing the expression of CD271, wherein
the chemotherapeutic agent is optionally selected from the group consisting of: dacarbazine, carmustine, cisplatin;
the non-steroidal anti-inflammatory agent is optionally selected from the group consisting of: ibuprofen, dexibuprofen, naproxen, fenoprofen, ketoprofen, dexketoprofen, flurbiprofen, oxaprozin, loxoprofen and mixtures thereof;
the immunotherapeutic agent is optionally selected from ipilimumab, nivolumab, pembrolizumab; and
the treatments increasing the expression of CD271 is are optionally selected from the BRAF/MET/KIT inhibitors.

23. A combination comprising:
a. a peptide of claim 1; and
b. a chemotherapeutic agent and/or non-steroidal anti-inflammatory agent, and/or immunotherapeutic agent and/or any treatment increasing the expression of CD271, wherein
the chemotherapeutic agent is optionally selected from the group consisting of: dacarbazine, carmustine, cisplatin;
the non-steroidal anti-inflammatory agent is optionally selected from the group consisting of: ibuprofen, dexibuprofen, naproxen, fenoprofen, ketoprofen, dexketoprofen, flurbiprofen, oxaprozin, loxoprofen and mixtures thereof and/or mixtures of any treatments increasing the expression of CD271;
the immunotherapeutic agent is optionally selected from ipilimumab, nivolumab, pembrolizumab; and
the treatments increasing the expression of CD271 are optionally selected from the BRAF/MET/KIT inhibitors.

24. A pharmaceutical composition comprising the combination of claim 23 and at least one pharmaceutically acceptable excipient and/or carrier, optionally in the form of an injectable pharmaceutical formulation.


SEQUENCE LISTING (5 sequences)

SEQ ID NO: 1 - protein, 11 aa, Artificial Sequence
  peptide
  MISC_FEATURE (1)..(1): X is selected from the group consisting of Met, Ala, Val, Ile, Leu, Phe, Tyr and Trp
  MISC_FEATURE (2)..(2): X is selected from the group consisting of Leu, Met, Ala, Val, Ile and Phe
  MISC_FEATURE (4)..(4): X is selected from the group consisting of Ile, Met, Ala, Val, Leu and Phe
  MISC_FEATURE (5)..(5): X is selected from the group consisting of Ile, Met, Ala, Val, Leu and Phe
  MISC_FEATURE (6)..(6): X is selected from the group consisting of Ala, Met, Val, Ile, Leu and Phe
  MISC_FEATURE (8)..(8): X is selected from the group consisting of Lys and Arg
  MISC_FEATURE (9)..(9): X is selected from the group consisting of Asn, Gln, His, Ser, Thr, Tyr and Cys
  MISC_FEATURE (10)..(10): X is selected from the group consisting of Ser, Asn, Gln, His, Thr, Tyr and Cys
XXGXXXGXXXG

SEQ ID NO: 2 - protein, 11 aa, Artificial Sequence
  peptide
  MISC_FEATURE (2)..(2): X is selected from the group consisting of Ser, Asn, Gln, His, Thr, Tyr and Cys
  MISC_FEATURE (3)..(3): X is selected from the group consisting of Asn, Gln, His, Ser, Thr, Tyr and Cys
  MISC_FEATURE (4)..(4): X is selected from the group consisting of Lys and Arg
  MISC_FEATURE (6)..(6): X is selected from the group consisting of Ala, Met, Val, Ile, Leu and Phe
  MISC_FEATURE (7)..(7): X is selected from the group consisting of Ile, Met, Ala, Val, Leu and Phe
  MISC_FEATURE (8)..(8): X is selected from the group consisting of Ile, Met, Ala, Val, Leu and Phe
  MISC_FEATURE (10)..(10): X is selected from the group consisting of Leu, Met, Ala, Val, Ile and Phe
  MISC_FEATURE (11)..(11): X is selected from the group consisting of Met, Ala, Val, Ile, Leu, Phe, Tyr and Trp
GXXXGXXXGXX

SEQ ID NO: 3 - protein, 11 aa, Artificial Sequence
  peptide
  MISC_FEATURE (1)..(1): X is DMet
  MISC_FEATURE (2)..(2): X is DLeu
  MISC_FEATURE (4)..(5): X is DIle
  MISC_FEATURE (6)..(6): X is DAla
  MISC_FEATURE (8)..(8): X is DLys
  MISC_FEATURE (9)..(9): X is DAsn
  MISC_FEATURE (10)..(10): X is DSer
XXGXXXGXXXG

SEQ ID NO: 4 - protein, 12 aa, Artificial Sequence
  peptide
  MISC_FEATURE (1)..(1): X is DMet
  MISC_FEATURE (2)..(2): X is DLeu
  MISC_FEATURE (4)..(5): X is DIle
  MISC_FEATURE (6)..(6): X is DAla
  MISC_FEATURE (8)..(8): X is DLys
  MISC_FEATURE (9)..(9): X is DAsn
  MISC_FEATURE (10)..(10): X is DSer
  MISC_FEATURE (12)..(12): X is DCys
XXGXXXGXXXGX

SEQ ID NO: 5 - protein, 12 aa, Artificial Sequence
  peptide
  MOD_RES (1)..(1): ACETYLATION
  MISC_FEATURE (1)..(1): X is DMet
  MISC_FEATURE (2)..(2): X is DLeu
  MISC_FEATURE (4)..(5): X is DIle
  MISC_FEATURE (6)..(6): X is DAla
  MISC_FEATURE (8)..(8): X is DLys
  MISC_FEATURE (9)..(9): X is DAsn
  MISC_FEATURE (10)..(10): X is DSer
  MOD_RES (12)..(12): AMIDATION
  MISC_FEATURE (12)..(12): X is DCys
  MOD_RES (12)..(12): AMIDATION
XXGXXXGXXXGX